SEQUENCE LISTING (114 sequences)

SEQ ID NO: 1 - protein, 235 aa, Homo sapiens
  REGION 1..235: note = misc_feature - Human CD8a, precursor (signal sequence is amino acids 1-21)
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 120 aa, synthetic construct
  REGION 1..120: note = Synthetic: B7 VHH
  VARIANT 119..120: note = Each X is G or is absent
QVQLVQSGGGLVRPGGSLRLSCAASGFTFDDYAMSWVRQAPGKGLEWVSTITWDGEGTDYAESMKGRFTISRDNAKSTVYLQMNGLKSEDTAVYVCAKGSPELQYDSWGQGTQVTVKPXX

SEQ ID NO: 3 - protein, 10 aa, synthetic construct
  REGION 1..10: note = Synthetic: CDR1 of B7, hzB7v1, hzB7v2, hzB7v3, hzB7v4, hzB7v5, hzB7v6, hzB7v7, hzB7v8, hzB7v9, hzB7v10, hzB7v11, hzB7v12, hzB7v13, hzB7v14, hzB7v15, hzB7v16, hzB7v17 hzB7v18, hzB7v19, hzB7v20, hzB7v21, hzB7v25, hzB7v26, hzB7v27, hzB7v28, hzB7v29, hzB7v30, hzB7v31, hzB7v33, hzB7v34, hzB7v35, hzB7v36, hzB7v37, hzB7v39, and hzB7v40
GFTFDDYAMS

SEQ ID NO: 4 - protein, 10 aa, synthetic construct
  REGION 1..10: note = Synthetic: CDR2 of B7, hzB7v1, hzB7v2, hzB7v3, hzB7v4, hzB7v5, hzB7v8, hzB7v9, hzB7v13, and hzB7v14
TITWDGEGTD

SEQ ID NO: 5 - protein, 9 aa, synthetic construct
  REGION 1..9: note = Synthetic: CDR3 of B7, hzB7v1, hzB7v2, hzB7v3, hzB7v4, hzB7v5, hzB7v6, hzB7v7, hzB7v10, hzB7v11, hzB7v12, hzB7v16, hzB7v17, and hzB7v18
GSPELQYDS

SEQ ID NO: 6 - protein, 120 aa, synthetic construct
  REGION 1..120: note = Synthetic: hzB7v1 VHH
  VARIANT 119..120: note = Each X is G or is absent
EVQLVESGGGEVQPGGSLRLSCAASGFTFDDYAMSWVRQAPGKGLEWVSTITWDGEGTDYAESVKGRFTISRDNAKNTLYLQMSSLRAEDTAVYYCAKGSPELQYDSWGQGTLVTVKPXX

SEQ ID NO: 7 - protein, 120 aa, synthetic construct
  REGION 1..120: note = Synthetic: hzB7v2 VHH
  VARIANT 119..120: note = Each X is G or is absent
EVQLVESGGGEVQPGGSLRLSCAASGFTFDDYAMSWVRQAPGKGLEWVSTITWDGEGTDYAESVKGRFTISRDNAKNTVYLQMSSLRAEDTAVYYCAKGSPELQYDSWGQGTLVTVKPXX

SEQ ID NO: 8 - protein, 120 aa, synthetic construct
  REGION 1..120: note = Synthetic: hzB7v3 VHH
  VARIANT 119..120: note = Each X is G or is absent
EVQLVESGGGEVQPGGSLRLSCAASGFTFDDYAMSWVRQAPGKGLEWVSTITWDGEGTDYAESMKGRFTISRDNAKNTVYLQMSSLRAEDTAVYYCAKGSPELQYDSWGQGTLVTVKPXX

SEQ ID NO: 9 - protein, 120 aa, synthetic construct
  REGION 1..120: note = Synthetic: hzB7v4 VHH
  VARIANT 119..120: note = Each X is G or is absent
EVQLVESGGGEVQPGGSLRLSCAASGFTFDDYAMSWVRQAPGKGLEWVSTITWDGEGTDYAESMKGRFTISRDNAKSTVYLQMSSLRAEDTAVYYCAKGSPELQYDSWGQGTLVTVKPXX

SEQ ID NO: 10 - protein, 120 aa, synthetic construct
  REGION 1..120: note = Synthetic: hzB7v5 VHH
  VARIANT 119..120: note = Each X is G or is absent
EVQLVESGGGEVQPGGSLRLSCAASGFTFDDYAMSWVRQAPGKGLEWVSTITWDGEGTDYAESMKGRFTISRDNAKSTVYLQMSSLRAEDTAVYVCAKGSPELQYDSWGQGTLVTVKPXX

SEQ ID NO: 11 - protein, 120 aa, synthetic construct
  REGION 1..120: note = Synthetic: hzB7v6 VHH
  VARIANT 119..120: note = Each X is G or is absent
EVQLVESGGGEVQPGGSLRLSCAASGFTFDDYAMSWVRQAPGKGLEWVSTITWEGEGTDYAESMKGRFTISRDNAKSTVYLQMSSLRAEDTAVYVCAKGSPELQYDSWGQGTLVTVKPXX

SEQ ID NO: 12 - protein, 10 aa, synthetic construct
  REGION 1..10: note = Synthetic: CDR2 of hzB7v6 and hzB7v10
TITWEGEGTD

SEQ ID NO: 13 - protein, 120 aa, synthetic construct
  REGION 1..120: note = Synthetic: hzB7v7 VHH
  VARIANT 119..120: note = Each X is G or is absent
EVQLVESGGGEVQPGGSLRLSCAASGFTFDDYAMSWVRQAPGKGLEWVSTITWDAEGTDYAESMKGRFTISRDNAKSTVYLQMSSLRAEDTAVYVCAKGSPELQYDSWGQGTLVTVKPXX

SEQ ID NO: 14 - protein, 10 aa, synthetic construct
  REGION 1..10: note = Synthetic: CDR2 of hzB7v7, hzB7v11, hzB7v15, hzB7v19, hzB7v20, hzB7v21, hzB7v23, hzB7v24, and hzB7v28
TITWDAEGTD

SEQ ID NO: 15 - protein, 120 aa, synthetic construct
  REGION 1..120: note = Synthetic: hzB7v8 VHH
  VARIANT 119..120: note = Each X is G or is absent
EVQLVESGGGEVQPGGSLRLSCAASGFTFDDYAMSWVRQAPGKGLEWVSTITWDGEGTDYAESMKGRFTISRDNAKSTVYLQMSSLRAEDTAVYVCAKGSPELQYESWGQGTLVTVKPXX

SEQ ID NO: 16 - protein, 9 aa, synthetic construct
  REGION 1..9: note = Synthetic: CDR3 of hzB7v8 and hzB7v13
GSPELQYES

SEQ ID NO: 17 - protein, 120 aa, synthetic construct
  REGION 1..120: note = Synthetic: hzB7v9 VHH
  VARIANT 119..120: note = Each X is G or is absent
EVQLVESGGGEVQPGGSLRLSCAASGFTFDDYAMSWVRQAPGKGLEWVSTITWDGEGTDYAESMKGRFTISRDNAKSTVYLQMSSLRAEDTAVYVCAKGSPELQYDTWGQGTLVTVKPXX

SEQ ID NO: 18 - protein, 9 aa, synthetic construct
  REGION 1..9: note = Synthetic: CDR3 of hzB7v9, hzB7v14, hzB7v15, hzB7v19, hzB7v20, hzB7v21, hzB7v23, hzB7v24, hzB7v25, hzB7v26, hzB7v27, hzB7v28, hzB7v29, hzB7v30, hzB7v31, hzB7v33, hzB7v34, hzB7v35, hzB7v36, hzB7v37, hzB7v39, and hzB7v40
GSPELQYDT

SEQ ID NO: 19 - protein, 120 aa, synthetic construct
  REGION 1..120: note = Synthetic: hzB7v10 VHH
EVQLVESGGGEVQPGGSLRLSCAASGFTFDDYAMSWVRQAPGKGLEWVSTITWEGEGTDYAESVKGRFTISRDNAKNTVYLQMSSLRAEDTAVYYCAKGSPELQYDSWGQGTLVTVKPGG

SEQ ID NO: 20 - protein, 120 aa, synthetic construct
  REGION 1..120: note = Synthetic: hzB7v11 VHH
  VARIANT 119..120: note = Each X is G or is absent
EVQLVESGGGEVQPGGSLRLSCAASGFTFDDYAMSWVRQAPGKGLEWVSTITWDAEGTDYAESVKGRFTISRDNAKNTVYLQMSSLRAEDTAVYYCAKGSPELQYDSWGQGTLVTVKPXX

SEQ ID NO: 21 - protein, 120 aa, synthetic construct
  REGION 1..120: note = Synthetic: hzB7v12 VHH
  VARIANT 119..120: note = Each X is G or is absent
EVQLVESGGGEVQPGGSLRLSCAASGFTFDDYAMSWVRQAPGKGLEWVSTITWSGEGTDYAESVKGRFTISRDNAKNTVYLQMSSLRAEDTAVYYCAKGSPELQYDSWGQGTLVTVKPXX

SEQ ID NO: 22 - protein, 10 aa, synthetic construct
  REGION 1..10: note = Synthetic: CDR2 of hzB7v12
TITWSGEGTD

SEQ ID NO: 23 - protein, 120 aa, synthetic construct
  REGION 1..120: note = Synthetic: hzB7v13 VHH
  VARIANT 119..120: note = Each X is G or is absent
EVQLVESGGGEVQPGGSLRLSCAASGFTFDDYAMSWVRQAPGKGLEWVSTITWDGEGTDYAESVKGRFTISRDNAKNTVYLQMSSLRAEDTAVYYCAKGSPELQYESWGQGTLVTVKPXX

SEQ ID NO: 24 - protein, 120 aa, synthetic construct
  REGION 1..120: note = Synthetic: hzB7v14 VHH
  VARIANT 119..120: note = Each X is G or is absent
EVQLVESGGGEVQPGGSLRLSCAASGFTFDDYAMSWVRQAPGKGLEWVSTITWDGEGTDYAESVKGRFTISRDNAKNTVYLQMSSLRAEDTAVYYCAKGSPELQYDTWGQGTLVTVKPXX

SEQ ID NO: 25 - protein, 120 aa, synthetic construct
  REGION 1..120: note = Synthetic: hzB7v15 VHH
  VARIANT 119..120: note = Each X is G or is absent
EVQLVESGGGEVQPGGSLRLSCAASGFTFDDYAMSWVRQAPGKGLEWVSTITWDAEGTDYAESVKGRFTISRDNAKNTVYLQMSSLRAEDTAVYYCAKGSPELQYDTWGQGTLVTVKPXX

SEQ ID NO: 26 - protein, 120 aa, synthetic construct
  REGION 1..120: note = Synthetic: hzB7v16 VHH
  VARIANT 119..120: note = Each X is G or is absent
EVQLVESGGGEVQPGGSLRLSCAASGFTFDDYAMSWVRQAPGKGLEWVSTITWEGESTDYAESVKGRFTISRDNAKNTVYLQMSSLRAEDTAVYYCAKGSPELQYDSWGQGTLVTVKPXX

SEQ ID NO: 27 - protein, 10 aa, synthetic construct
  REGION 1..10: note = Synthetic: CDR2 of hzB7v16
TITWEGESTD

SEQ ID NO: 28 - protein, 120 aa, synthetic construct
  REGION 1..120: note = Synthetic: hzB7v17 VHH
EVQLVESGGGEVQPGGSLRLSCAASGFTFDDYAMSWVRQAPGKGLEWVSTITWEGEGTYYAESVKGRFTISRDNAKNTVYLQMSSLRAEDTAVYYCAKGSPELQYDSWGQGTLVTVKPGG

SEQ ID NO: 29 - protein, 10 aa, synthetic construct
  REGION 1..10: note = Synthetic: CDR2 of hzB7v17
TITWEGEGTY

SEQ ID NO: 30 - protein, 120 aa, synthetic construct
  REGION 1..120: note = Synthetic: hzB7v18 VHH
  VARIANT 119..120: note = Each X is G or is absent
EVQLVESGGGEVQPGGSLRLSCAASGFTFDDYAMSWVRQAPGKGLEWVSTITWEGESTYYAESVKGRFTISRDNAKNTVYLQMSSLRAEDTAVYYCAKGSPELQYDSWGQGTLVTVKPXX

SEQ ID NO: 31 - protein, 10 aa, synthetic construct
  REGION 1..10: note = Synthetic: CDR2 of hzB7v18
TITWEGESTY

SEQ ID NO: 32 - protein, 227 aa, Homo sapiens
  REGION 1..227: note = misc_feature - human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 224 aa, Homo sapiens
  REGION 1..224: note = misc_feature - human IgG1 Fc xELL
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 227 aa, synthetic construct
  REGION 1..227: note = Synthetic: Fc region M252Y and M428V (YV) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 35 - protein, 227 aa, synthetic construct
  REGION 1..227: note = Synthetic: Fc region M252Y, M428V, H435R (YVR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 36 - protein, 224 aa, synthetic construct
  REGION 1..224: note = Synthetic: Fc region xELL H435R
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 37 - protein, 224 aa, synthetic construct
  REGION 1..224: note = Synthetic: Fc region xELL M252Y and M428V (YV)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPGK

SEQ ID NO: 38 - protein, 224 aa, synthetic construct
  REGION 1..224: note = Synthetic: Fc region xELL M252Y and M428L (YL)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 39 - protein, 224 aa, synthetic construct
  REGION 1..224: note = Synthetic: Fc region xELL M252Y, M428L, H435R (YLR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPGK

SEQ ID NO: 40 - protein, 224 aa, synthetic construct
  REGION 1..224: note = Synthetic: Fc region xELL M252Y, M428V, H435R (YVR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPGK

SEQ ID NO: 41 - protein, 224 aa, synthetic construct
  REGION 1..224: note = Synthetic: Fc region xELL S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 224 aa, synthetic construct
  REGION 1..224: note = Synthetic: Fc region xELL H435R S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 43 - protein, 224 aa, synthetic construct
  REGION 1..224: note = Synthetic: Fc region xELL M252Y and M428V (YV) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 44 - protein, 224 aa, synthetic construct
  REGION 1..224: note = Synthetic: Fc region xELL M252Y and M428L (YL) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 45 - protein, 224 aa, synthetic construct
  REGION 1..224: note = Synthetic: Fc region xELL M252Y, M428L, H435R (YLR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPGK

SEQ ID NO: 46 - protein, 224 aa, synthetic construct
  REGION 1..224: note = Synthetic: Fc region xELL M252Y, M428V, H435R (YVR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPGK

SEQ ID NO: 47 - protein, 224 aa, synthetic construct
  REGION 1..224: note = Synthetic: Fc region xELL T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 224 aa, synthetic construct
  REGION 1..224: note = Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 49 - protein, 224 aa, synthetic construct
  REGION 1..224: note = Synthetic: Fc region xELL M252Y and M428V (YV) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPGK

SEQ ID NO: 50 - protein, 224 aa, synthetic construct
  REGION 1..224: note = Synthetic: Fc region xELL M252Y and M428L (YL) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 51 - protein, 224 aa, synthetic construct
  REGION 1..224: note = Synthetic: Fc region xELL M252Y, M428L, H435R (YLR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPGK

SEQ ID NO: 52 - protein, 224 aa, synthetic construct
  REGION 1..224: note = Synthetic: Fc region xELL M252Y, M428V, H435R (YVR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 53 - protein, 227 aa, synthetic construct
  REGION 1..227: note = Synthetic: Fc region H435R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 54 - protein, 227 aa, synthetic construct
  REGION 1..227: note = Synthetic: Fc region M252Y and M428V (YV)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPGK

SEQ ID NO: 55 - protein, 227 aa, synthetic construct
  REGION 1..227: note = Synthetic: Fc region M252Y and M428L (YL)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 56 - protein, 227 aa, synthetic construct
  REGION 1..227: note = Synthetic: Fc region M252Y, M428L, H435R (YLR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPGK

SEQ ID NO: 57 - protein, 227 aa, synthetic construct
  REGION 1..227: note = Synthetic: Fc region M252Y, M428V, H435R (YVR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPGK

SEQ ID NO: 58 - protein, 227 aa, synthetic construct
  REGION 1..227: note = Synthetic: Fc region S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 227 aa, synthetic construct
  REGION 1..227: note = Synthetic: Fc region H435R S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 60 - protein, 227 aa, synthetic construct
  REGION 1..227: note = Synthetic: Fc region M252Y and M428L (YL) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 61 - protein, 227 aa, synthetic construct
  REGION 1..227: note = Synthetic: Fc region M252Y, M428L, H435R (YLR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPGK

SEQ ID NO: 62 - protein, 227 aa, synthetic construct
  REGION 1..227: note = Synthetic: Fc region M252Y, M428V, H435R (YVR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPGK

SEQ ID NO: 63 - protein, 227 aa, synthetic construct
  REGION 1..227: note = Synthetic: Fc region T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 227 aa, synthetic construct
  REGION 1..227: note = Synthetic: Fc region H435R, T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 65 - protein, 227 aa, synthetic construct
  REGION 1..227: note = Synthetic: Fc region M252Y and M428V (YV) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPGK

SEQ ID NO: 66 - protein, 227 aa, synthetic construct
  REGION 1..227: note = Synthetic: Fc region M252Y and M428L (YL) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 67 - protein, 227 aa, synthetic construct
  REGION 1..227: note = Synthetic: Fc region M252Y, M428L, H435R (YLR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPGK

SEQ ID NO: 68 - protein, 223 aa, synthetic construct
  REGION 1..223: note = Synthetic: Fc region xELL P329G, H435R, T366S, L368A, Y407V hole deltaK447
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 69 - protein, 223 aa, synthetic construct
  REGION 1..223: note = Synthetic: Fc region xELL P329G, S354C, T366W knob deltaK447
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 223 aa, synthetic construct
  REGION 1..223: note = Synthetic: Fc region IgG1 xELL S354C, T366W knobdeltaK447
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 133 aa, Homo sapiens [sequence omitted]

SEQ ID NO: 72 - protein, 368 aa, synthetic construct
  REGION 1..368: note = Synthetic: CD8a-IgV LFc antigen for immunization (signal sequence is amino acids 1-21)
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKTGGSGGGGCPPCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSLGK

SEQ ID NO: 73 - protein, 10 aa, synthetic construct
  REGION 1..10: note = Synthetic: CDR1 of hzB7v23
GFTFDSYAMS

SEQ ID NO: 74 - protein, 10 aa, synthetic construct
  REGION 1..10: note = Synthetic: CDR1 of hzB7v24
GFTFSSYAMS

SEQ ID NO: 75 - protein, 10 aa, synthetic construct
  REGION 1..10: note = Synthetic: CDR2 of hzB7v25
TITWSAEGTD

SEQ ID NO: 76 - protein, 10 aa, synthetic construct
  REGION 1..10: note = Synthetic: CDR2 of hzB7v26, hzB7v30, and hzB7v36
TITWDAGGTD

SEQ ID NO: 77 - protein, 10 aa, synthetic construct
  REGION 1..10: note = Synthetic: CDR2 of hzB7v27, hzB7v29, and hzB7v35
TITWDAEGTY

SEQ ID NO: 78 - protein, 10 aa, synthetic construct
  REGION 1..10: note = Synthetic: CDR2 of hzB7v31 and hzB7v37
TITWDAGGTY

SEQ ID NO: 79 - protein, 10 aa, synthetic construct
  REGION 1..10: note = Synthetic: CDR2 of hzB7v33 and hzB7v39
TITWSAEGTY

SEQ ID NO: 80 - protein, 10 aa, synthetic construct
  REGION 1..10: note = Synthetic: CDR2 of hzB7v34 and hzB7v40
TITWSAGGTD

SEQ ID NO: 81 - protein, 118 aa, synthetic construct
  REGION 1..118: note = Synthetic: hzB7v19 VHH
QVQLVESGGGEVQPGGSLRLSCAASGFTFDDYAMSWVRQAPGKGLEWVSTITWDAEGTDYAESVKGRFTISRDNAKNTVYLQMSSLRAEDTAVYYCAKGSPELQYDTWGQGTLVTVKP

SEQ ID NO: 82 - protein, 118 aa, synthetic construct
  REGION 1..118: note = Synthetic: hzB7v20 VHH
EVQLVQSGGGEVQPGGSLRLSCAASGFTFDDYAMSWVRQAPGKGLEWVSTITWDAEGTDYAESVKGRFTISRDNAKNTVYLQMSSLRAEDTAVYYCAKGSPELQYDTWGQGTLVTVKP

SEQ ID NO: 83 - protein, 118 aa, synthetic construct
  REGION 1..118: note = Synthetic: hzB7v21 VHH
QVQLVQSGGGEVQPGGSLRLSCAASGFTFDDYAMSWVRQAPGKGLEWVSTITWDAEGTDYAESVKGRFTISRDNAKNTVYLQMSSLRAEDTAVYYCAKGSPELQYDTWGQGTLVTVKP

SEQ ID NO: 84 - protein, 118 aa, synthetic construct
  REGION 1..118: note = Synthetic: hzB7v23 VHH
QVQLVQSGGGEVQPGGSLRLSCAASGFTFDSYAMSWVRQAPGKGLEWVSTITWDAEGTDYAESVKGRFTISRDNAKNTVYLQMSSLRAEDTAVYYCAKGSPELQYDTWGQGTLVTVKP

SEQ ID NO: 85 - protein, 118 aa, synthetic construct
  REGION 1..118: note = Synthetic: hzB7v24 VHH
QVQLVQSGGGEVQPGGSLRLSCAASGFTFSSYAMSWVRQAPGKGLEWVSTITWDAEGTDYAESVKGRFTISRDNAKNTVYLQMSSLRAEDTAVYYCAKGSPELQYDTWGQGTLVTVKP

SEQ ID NO: 86 - protein, 118 aa, synthetic construct
  REGION 1..118: note = Synthetic: hzB7v25 VHH
QVQLVQSGGGEVQPGGSLRLSCAASGFTFDDYAMSWVRQAPGKGLEWVSTITWSAEGTDYAESVKGRFTISRDNAKNTVYLQMSSLRAEDTAVYYCAKGSPELQYDTWGQGTLVTVKP

SEQ ID NO: 87 - protein, 118 aa, synthetic construct
  REGION 1..118: note = Synthetic: hzB7v26 VHH
QVQLVQSGGGEVQPGGSLRLSCAASGFTFDDYAMSWVRQAPGKGLEWVSTITWDAGGTDYAESVKGRFTISRDNAKNTVYLQMSSLRAEDTAVYYCAKGSPELQYDTWGQGTLVTVKP

SEQ ID NO: 88 - protein, 118 aa, synthetic construct
  REGION 1..118: note = Synthetic: hzB7v27 VHH
QVQLVQSGGGEVQPGGSLRLSCAASGFTFDDYAMSWVRQAPGKGLEWVSTITWDAEGTYYAESVKGRFTISRDNAKNTVYLQMSSLRAEDTAVYYCAKGSPELQYDTWGQGTLVTVKP

SEQ ID NO: 89 - protein, 118 aa, synthetic construct
  REGION 1..118: note = Synthetic: hzB7v28 VHH
QVQLVQSGGGEVQPGGSLRLSCAASGFTFDDYAMSWVRQAPGKGLEWVSTITWDAEGTDYAAPVKGRFTISRDNAKNTVYLQMSSLRAEDTAVYYCAKGSPELQYDTWGQGTLVTVKP

SEQ ID NO: 90 - protein, 118 aa, synthetic construct
  REGION 1..118: note = Synthetic: hzB7v29 VHH
QVQLVQSGGGEVQPGGSLRLSCAASGFTFDDYAMSWVRQAPGKGLEWVSTITWDAEGTYYAAPVKGRFTISRDNAKNTVYLQMSSLRAEDTAVYYCAKGSPELQYDTWGQGTLVTVKP

SEQ ID NO: 91 - protein, 118 aa, synthetic construct
  REGION 1..118: note = Synthetic: hzB7v30 VHH
QVQLVQSGGGEVQPGGSLRLSCAASGFTFDDYAMSWVRQAPGKGLEWVSTITWDAGGTDYAAPVKGRFTISRDNAKNTVYLQMSSLRAEDTAVYYCAKGSPELQYDTWGQGTLVTVKP

SEQ ID NO: 92 - protein, 118 aa, synthetic construct
  REGION 1..118: note = Synthetic: hzB7v31 VHH
QVQLVQSGGGEVQPGGSLRLSCAASGFTFDDYAMSWVRQAPGKGLEWVSTITWDAGGTYYAAPVKGRFTISRDNAKNTVYLQMSSLRAEDTAVYYCAKGSPELQYDTWGQGTLVTVKP

SEQ ID NO: 93 - protein, 118 aa, synthetic construct
  REGION 1..118: note = Synthetic: hzB7v33 VHH
QVQLVQSGGGEVQPGGSLRLSCAASGFTFDDYAMSWVRQAPGKGLEWVSTITWSAEGTYYAAPVKGRFTISRDNAKNTVYLQMSSLRAEDTAVYYCAKGSPELQYDTWGQGTLVTVKP

SEQ ID NO: 94 - protein, 118 aa, synthetic construct
  REGION 1..118: note = Synthetic: hzB7v34 VHH
QVQLVQSGGGEVQPGGSLRLSCAASGFTFDDYAMSWVRQAPGKGLEWVSTITWSAGGTDYAAPVKGRFTISRDNAKNTVYLQMSSLRAEDTAVYYCAKGSPELQYDTWGQGTLVTVKP

SEQ ID NO: 95 - protein, 118 aa, synthetic construct
  REGION 1..118: note = Synthetic: hzB7v36 VHH
QVQLVQSGGGEVKPGGSLRLSCAASGFTFDDYAMSWVRQAPGKGLEWVSTITWDAGGTDYAAPVKGRFTISRDNAKNTVYLQMSSLRAEDTAVYYCAKGSPELQYDTWGQGTLVTVKP

SEQ ID NO: 96 - protein, 118 aa, synthetic construct
  REGION 1..118: note = Synthetic: hzB7v37 VHH
QVQLVQSGGGEVKPGGSLRLSCAASGFTFDDYAMSWVRQAPGKGLEWVSTITWDAGGTYYAAPVKGRFTISRDNAKNTVYLQMSSLRAEDTAVYYCAKGSPELQYDTWGQGTLVTVKP

SEQ ID NO: 97 - protein, 118 aa, synthetic construct
  REGION 1..118: note = Synthetic: hzB7v39 VHH
QVQLVQSGGGEVKPGGSLRLSCAASGFTFDDYAMSWVRQAPGKGLEWVSTITWSAEGTYYAAPVKGRFTISRDNAKNTVYLQMSSLRAEDTAVYYCAKGSPELQYDTWGQGTLVTVKP

SEQ ID NO: 98 - protein, 118 aa, synthetic construct
  REGION 1..118: note = Synthetic: hzB7v40 VHH
QVQLVQSGGGEVKPGGSLRLSCAASGFTFDDYAMSWVRQAPGKGLEWVSTITWSAGGTDYAAPVKGRFTISRDNAKNTVYLQMSSLRAEDTAVYYCAKGSPELQYDTWGQGTLVTVKP

SEQ ID NO: 99 - protein, 118 aa, synthetic construct
  REGION 1..118: note = Synthetic: hzB7v35 VHH
QVQLVQSGGGEVKPGGSLRLSCAASGFTFDDYAMSWVRQAPGKGLEWVSTITWDAEGTYYAAPVKGRFTISRDNAKNTVYLQMSSLRAEDTAVYYCAKGSPELQYDTWGQGTLVTVKP

SEQ ID NO: 100 - protein, 118 aa, synthetic construct
  REGION 1..118: note = Synthetic: hzB7v41 VHH
QVQLVQSGGGEVQPGGSLRLSCAASGFTFDDYAMSWVRQAPGKGLEWVSTITWDAGGTYYAAPVKGRFTISRDNAKNTVYLQMSSLRAEDTAVYYCAKGSPELQYDTWGQGTLVTVRP

SEQ ID NO: 101 - protein, 223 aa, synthetic construct
  REGION 1..223: note = Synthetic: Fc region xELL H435R, T366S, L368A, Y407V hole deltaK447
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 102 - protein, 226 aa, synthetic construct
  REGION 1..226: note = Synthetic: Fc region S354C T366W knob, deltaK447
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 226 aa, synthetic construct
  REGION 1..226: note = Synthetic: Fc region H435R S354C T366W knob, deltaK447
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPG

SEQ ID NO: 104 - protein, 226 aa, synthetic construct
  REGION 1..226: note = Synthetic: Fc region M252Y and M428L (YL) S354C T366W knob, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPG

SEQ ID NO: 105 - protein, 226 aa, synthetic construct
  REGION 1..226: note = Synthetic: Fc region M252Y, M428L, H435R (YLR) S354C T366W knob, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPG

SEQ ID NO: 106 - protein, 226 aa, synthetic construct
  REGION 1..226: note = Synthetic: Fc region M252Y, M428V, H435R (YVR) S354C T366W knob, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPG

SEQ ID NO: 107 - protein, 226 aa, synthetic construct
  REGION 1..226: note = Synthetic: Fc region T366S, L368A, Y407V hole, deltaK447
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 108 - protein, 226 aa, synthetic construct
  REGION 1..226: note = Synthetic: Fc region H435R, T366S, L368A, Y407V hole, deltaK447
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 109 - protein, 226 aa, synthetic construct
  REGION 1..226: note = Synthetic: Fc region M252Y and M428V (YV) T366S, L368A, Y407V hole, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPG

SEQ ID NO: 110 - protein, 226 aa, synthetic construct
  REGION 1..226: note = Synthetic: Fc region M252Y and M428L (YL) T366S, L368A, Y407V hole, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPG

SEQ ID NO: 111 - protein, 226 aa, synthetic construct
  REGION 1..226: note = Synthetic: Fc region M252Y, M428L, H435R (YLR) T366S, L368A, Y407V hole, deltaK447
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPG

SEQ ID NO: 112 - protein, 4 aa, synthetic construct
  REGION 1..4: note = Synthetic: Linker 1
GGGG

SEQ ID NO: 113 - protein, 6 aa, synthetic construct
  REGION 1..6: note = Synthetic: Linker 2
GGSGGS

SEQ ID NO: 114 - protein, 6 aa, synthetic construct
  REGION 1..6: note = Synthetic: Linker 3
GGSSGS

(19) United States
(12) Patent Application Publication
(10) Pub. No.: US 2025/0320295 A1
(43) Pub. Date: Oct. 16, 2025

(54) CD8-Binding Polypeptides and Uses Thereof

(71) Applicant: Inhibrx Biosciences, Inc., La Jolla, CA (US)

(72) Inventors: John C. Timmer, San Diego, CA (US); William Crago, San Diego, CA (US); Florian Sulzmaier, San Diego, CA (US); Lucas Rascon, San Diego, CA (US); Brendan P. Eckelman, Encinitas, CA (US)

(73) Assignee: Inhibrx Biosciences, Inc., La Jolla, CA (US)

(21) Appl. No.: 18/578,133

(22) PCT Filed: Jul. 19, 2022
(86) PCT No.: PCT/US2022/073877

§ 371 Date: Jan. 10, 2024

Related U.S. Application Data
(60) Provisional application No. 63/223,786, filed on Jul. 20, 2021.
(60) Provisional application No. 63/288,111, filed on Dec. 10, 2021.

(51) Int. Cl. C07K 16/28; A61K 39/00; A61K 47/68; A61P 35/00; C07K 14/55

(52) U.S. Cl. CPC C07K 16/2815; A61K 47/6803; A61K 47/68031; A61P 35/00; C07K 14/55; A61K 2039/505; C07K 2317/24; C07K 2317/52; C07K 2317/569; C07K 2317/732; C07K 2317/92; C07K 2319/00

(57) ABSTRACT

Provided herein are VHH-containing polypeptides that bind CD8. Uses of the VHH-containing polypeptides are also provided.

### CROSS-REFERENCE TO RELATED APPLICATIONS

This application claims the benefit of priority of U.S. Provisional Application No. 63/223,786, filed Jul. 20, 2021; and U.S. Provisional Application No. 63/288,111, filed Dec. 10, 2021; each of which is incorporated by reference herein in its entirety for any purpose.

### SEQUENCE LISTING

The present application contains a Sequence Listing, which has been submitted electronically in XML format. Said XML copy, was created on Sep. 13, 2024, is named “2022-09-13_01202-0031-00US_Sequence_lising,” and is 168,725 bytes in size. The information in the electronic format of the sequence listing is incorporated herein by reference in its entirety.

### FIELD

The present invention relates to CD8-binding polypeptides, and methods of using CD8-binding polypeptides to modulate the biological activity of CD8. Such methods include, but are not limited to, methods of treating cancer. In some embodiments, the CD8-binding polypeptides are fusion polypeptides comprising a CD8-binding polypeptide and a polypeptide that binds an antigen other than CD8.

### BACKGROUND

CD8 is a transmembrane glycoprotein expressed on the surface of cytotoxic T cells (CD8+ T cells), and also other cells of the lymphoid system, including natural killer cells, γδ T cells, cortical thymocytes, and subsets of dendritic cells. CD8 is typically a heterodimer composed of a CD8α chain and CD8β chain, but may in some circumstances exist as a CD8α homodimer. On cytotoxic T cells, CD8 acts as a co-receptor for the T-cell receptor (TCR) to enhance antigen recognition and T cell activation. Cytotoxic T cell activation is governed by the interaction of TCR with peptide antigen bound to class I major histocompatibility complex (MHC) proteins. CD8 helps stabilize the TCR/peptide-MHC interaction through binding to an invariant region of class I MHC proteins. CD8 also enhances TCR signaling by recruiting Lck to the cytoplasmic domain of CD8α leading to a cascade that amplifies T cell activation signals.

Activation of T cells is also controlled by other molecules, such as IL-2, IL-15, IL-7, IL-6, IL-12, IFNα, IFNβ, and IFNγ. The cytokine interleukin-2 (IL-2), which is synthesized and secreted by the activated T cell itself, is a pleiotropic cytokine that modulates differentiation of helper T cells, augments cytolytic activity of natural killer cells, and regulates CD8+ T cell generation. IL-2 binds to a high affinity receptor composed of three subunits (IL-2α, IL-2β, and γc) on the T cell surface. Signaling through the IL-2 receptor complex triggers the T cell to progress through cell division, driving clonal expansion of the activated T cell.

There exists a need for CD8-binding polypeptides that can specifically target activating molecules to CD8+ T cells to increase the potency and selectivity of cytotoxic T cell responses.

### SUMMARY

Provided herein are CD8-binding polypeptides, and methods of using CD8-binding polypeptides to treat, for example, cancer. In some embodiments, a CD8-binding polypeptide comprises one or more additional binding domains and/or cytokine sequences. Certain numbered embodiments are provided below.

Embodiment 1 A polypeptide comprising at least one VHH domain that binds CD8 and that comprises a CDR1 comprising the amino acid sequence of SEQ ID NO: 3, 73, or 74; a CDR2 comprising the amino acid sequence of SEQ ID NO: 4, 12, 14, 22, 27, 29, 31, 75, 76, 77, 78, 79, or 80; and a CDR3 comprising the amino acid sequence of SEQ ID NO: 5, 16, or 18.

Embodiment 2 The polypeptide of embodiment 1, wherein at least one VHH domain comprises a CDR1, a CDR2, and a CDR3, respectively comprising the amino acid sequences of SEQ ID NOs: 3, 4, and 5; 3, 12, and 5; 3, 14, and 5; 3, 4, and 16; 3, 4, and 18; 3, 22, and 5; 3, 14, and 18; 3, 27, and 5; 3, 29, and 5; 3, 31, and 5; 73, 14, and 18; 74, 14, and 18; 3, 75, and 18; 3, 76, and 18; 3, 77, and 18; 3, 78, and 18; 3, 79, and 18; or 3, 80 and 18.

Embodiment 3 The polypeptide of embodiment 1 or embodiment 2, wherein at least one VHH domain comprises a CDR1 comprising the amino acid sequence of SEQ ID NO: 3; a CDR2 comprising the amino acid sequence of SEQ ID NO: 78; and a CDR3 comprising the amino acid sequence of SEQ ID NO: 18.

Embodiment 4 The polypeptide of any one of embodiments 1-3, wherein at least one VHH domain, or each VHH domain, is humanized.

Embodiment 5 The polypeptide of any one of embodiment 1-4, wherein at least one VHH domain comprises an amino acid sequence at least 85%, 90%, 95%, or at least 99% identical to the amino acid sequence of SEQ ID NO: 2, 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, 99, or 100.

Embodiment 6 The polypeptide of any one of embodiments 1-5, wherein at least one VHH domain comprises the amino acid sequence of SEQ ID NO: 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, or 99, or 100.

Embodiment 7 The polypeptide of any one of embodiments 1-6, wherein at least one VHH domain comprises the amino acid sequence of SEQ ID NO: 92 or 100.

Embodiment 8 The polypeptide of any one of embodiments 1-7, comprising two VHH domains.

Embodiment 9 The polypeptide of any one of embodiments 1-7, comprising three VHH domains.

Embodiment 10 The polypeptide of any one of embodiments 1-9, wherein the polypeptide comprises an immune cell activating cytokine.

Embodiment 11 The polypeptide of embodiment 10, wherein the immune cell activating cytokine is fused to the N-terminus or C-terminus of a VHH domain that binds CD8. Embodiment 12 The polypeptide of embodiment 10 or embodiment 11, wherein the immune cell activating cytokine is IL-2, IL-15, IL-7, IL-6, IL-12, IFNα, IFNβ, or IFNγ, or an attenuated or modified version thereof.

Embodiment 13 The polypeptide of any one of embodiments 1-12, wherein the polypeptide comprises an Fc region.

Embodiment 14 The polypeptide of embodiment 13, wherein the Fc region comprises an amino acid sequence selected from SEQ ID NOs: 32-70, or 101-111.

Embodiment 15 The polypeptide of embodiment 13 or embodiment 14, wherein the polypeptide comprises an immune cell activating cytokine.

Embodiment 16 The polypeptide of embodiment 15, wherein the immune cell activating cytokine is IL-2, IL-15, IL-7, IL-6, IL-12, IFNα, IFNβ, or IFNγ, or an attenuated or modified version thereof

Embodiment 17 The polypeptide of embodiment 16, wherein the immune cell activating cytokine is fused to the C-terminus of the Fc region.

Embodiment 18 The polypeptide of any one of embodiments 1-17, wherein the polypeptide comprises at least one antigen-binding domain that binds an antigen other than CD8.

Embodiment 19 The polypeptide of embodiment 18, wherein the polypeptide comprises at least one antigen-binding domain that binds Lag3, CTLA4, TGFBR1, TGFBR2, Fas, TNFR2, PD1, PDL1, or TIM3.

Embodiment 20 The polypeptide of embodiment 18 or 19, wherein the polypeptide comprises at least one antigen-binding domain that binds TGFBR1, TGFBR2, Fas, TNFR2, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, PIGF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.

Embodiment 21 The polypeptide of any one of embodiments 18-20, wherein at least one antigen binding-domain that binds an antigen other than CD8 is a VHH domain.

Embodiment 22 The polypeptide of embodiment 21, wherein each antigen-binding domain that binds an antigen other than CD8 is a VHH domain.

Embodiment 23 The polypeptide of any one of embodiments 18-21, wherein at least one antigen-binding domain that binds an antigen other than CD8 comprises a heavy chain variable region and a light chain variable region.

Embodiment 24 The polypeptide of embodiment 23, wherein each antigen-binding domain that binds an antigen other than CD8 comprises a heavy chain variable region and a light chain variable region.

Embodiment 25 A complex comprising a first polypeptide and a second polypeptide, wherein the first polypeptide is the polypeptide of any one of embodiments 13-24, wherein the first polypeptide comprises a first Fc region, and wherein the second polypeptide comprises a second Fc region, and wherein the first and second Fc regions are the same or different.

Embodiment 26 The complex of embodiment 25, wherein the second polypeptide comprises at least one VHH domain that binds CD8, at least one immune cell activating cytokine, and/or at least one antigen binding domain that binds an antigen other than CD8.

Embodiment 27 The complex of embodiment 26, wherein if the antigen-binding domain that binds an antigen other than CD8 comprises a heavy chain variable region and a light chain variable region, then the heavy chain variable region is fused to a heavy chain constant region comprising the second Fc region.

Embodiment 28 The complex of any one of embodiments 25-27, wherein the first Fc region comprises a knob mutation and the second Fc region comprises a hole mutation.

Embodiment 29 The complex of embodiment 28, wherein the first Fc region comprises a T366W mutation and the second Fc region comprises T366S, L368A, and Y407V mutations.

Embodiment 30 The complex of embodiment 29, wherein the second Fc region comprises a H435R or H435K mutation.

Embodiment 31 The polypeptide or complex of any one of embodiments 13-30, wherein the polypeptide is a dimer under physiological conditions, or wherein the complex is formed under physiological conditions.

Embodiment 32 The polypeptide or complex of any one of embodiments 1-31, wherein the CD8 is human CD8.

Embodiment 33 The polypeptide or complex of embodiment 32, wherein the human CD8 comprises the sequence of SEQ ID NO: 1.

Embodiment 34 An immunoconjugate comprising the polypeptide or complex of any one of embodiments 1-33 and a cytotoxic agent.

Embodiment 35 The immunoconjugate of embodiment 34, wherein the cytotoxic agent is selected from a calicheamicin, an auristatin, a dolastatin, a tubulicin, a maytansinoid, a cryptophycin, a duocarmycin, an esperamicin, a pyrrolobenzodiazepine, and an enediyne antibiotic.

Embodiment 36 A pharmaceutical composition comprising the polypeptide or complex of any one of embodiments 1-33 or the immunoconjugate of embodiment 34 or embodiment 35, and a pharmaceutically acceptable carrier.

Embodiment 37 An isolated nucleic acid that encodes the polypeptide or complex of any one of embodiments 1-33.

Embodiment 38 A vector comprising the nucleic acid of embodiment 37.

Embodiment 39 A host cell comprising the nucleic acid of embodiment 37 or the vector of embodiment 38.

Embodiment 40 A host cell that expresses the polypeptide or complex of any one of embodiments 1-33.

Embodiment 41 A method of producing the polypeptide or complex of any one of embodiments 1-33, comprising incubating the host cell of embodiment 38 or embodiment 39 under conditions suitable for expression of the polypeptide or complex.

Embodiment 42 The method of embodiment 41, further comprising isolating the polypeptide or complex.

Embodiment 43 A method of increasing CD8 T cell proliferation comprising contacting T cells with the polypeptide or complex of any one of embodiments 1-33.

Embodiment 44 The method of embodiment 43, wherein the CD8 T cells are in vitro.

Embodiment 45 The method of embodiment 43, wherein the CD8 T cells are in vivo.

Embodiment 46 A method of treating cancer comprising administering to a subject with cancer a pharmaceutically effective amount of the polypeptide or complex of any one of embodiments 1-33, or the pharmaceutical composition of embodiment 36.

Embodiment 47 The method of embodiment 46, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

Embodiment 48 The method of embodiment 46 or 47, further comprising administering an additional therapeutic agent.

Embodiment 49 The method of embodiment 48, wherein the additional therapeutic agent is an anti-cancer agent.

Embodiment 50 The method of embodiment 49, wherein the anti-cancer agent is selected from a chemotherapeutic agent, an anti-cancer biologic, radiation therapy, CAR-T therapy, and an oncolytic virus.

Embodiment 51 The method of embodiment 48, wherein the additional therapeutic agent is an anti-cancer biologic.

Embodiment 52 The method of embodiment 51, wherein the anti-cancer biologic is an agent that inhibits PD-1 and/or PD-L1.

Embodiment 53 The method of embodiment 51, wherein the anti-cancer biologic is an agent that inhibits VISTA, gpNMB, B7H3, B7H4, HHLA2, CTLA4, or TIGIT.

Embodiment 54 The method of any one of embodiment 49, wherein the anti-cancer agent is an antibody.

Embodiment 55 The method of embodiment 51, wherein the anti-cancer biologic is a cytokine.

Embodiment 56 The method of embodiment 49, wherein the anti-cancer agent is CAR-T therapy.

Embodiment 57 The method of embodiment 49, wherein the anti-cancer agent is an oncolytic virus.

Embodiment 58 The method of any one of embodiments 46-57, further comprising tumor resection and/or radiation therapy.

### BRIEF DESCRIPTION OF THE FIGURES

FIG. 1A-1B show binding of CD8a-targeting sdAbs formatted as VHH-hIgG1-Fc fusion proteins as assessed by flow cytometry. FIG. 1A shows binding to isolated human T cells. FIG. 1B shows binding to HEK293FS cells as a CD8a-negative control.

FIG. 2A-2B show binding of CD8a-targeting sdAbs formatted as VHH-hIgG1-Fc fusion proteins as assessed by flow cytometry. FIG. 2A shows binding to isolated human T cells. FIG. 2B shows binding to HEK293FS cells as a CD8a-negative control.

FIG. 3A-3B show binding of CD8a-targeting sdAbs formatted as VHH-hIgG1-Fc fusion proteins as assessed by flow cytometry. FIG. 3A shows binding to human CD8a-FL cells (expressing full-length CD8a). FIG. 3B shows binding on cynomolgus CD8a-FL cells.

FIG. 4A-4B show binding of CD8a-targeting sdAbs formatted as VHH-hIgG1-Fc fusion proteins as assessed by flow cytometry. FIG. 4A shows binding to isolated human CD3+CD4− T cells. FIG. 4B shows binding to isolated cynomolgus CD3+CD4− peripheral blood mononuclear cells (PBMC).

FIG. 5A-5B show binding of the fusion protein CD8a-hzB7v15 xELL-Fc as assessed by flow cytometry. FIG. 5A shows binding to human CD3+CD4− Leuko 29 T cells. FIG. 5B shows binding to cynomolgus CD3+CD4−CD16− T cells.

FIG. 6A-6C show IL-2 activities of wild type IL-2 and CD8a-targeting VHH-hIgG1-fusion proteins comprising CD8a-hzB7v15 and an attenuated IL-2 on IL-2 reporter cells. FIG. 6A shows IL-2 activities on reporter cells that do not express CD8. FIG. 6B shows IL-2 activities on IL-2 reporter cells that do express CD8. FIG. 6C shows the activity of wild type IL-2, fusion proteins comprising CD8a-hzB7v31 and an attenuated IL-2 mutant, and a non-targeted attenuated IL-2 mutant comprising the same mutations on IL-2 reporter cells that express CD8.

FIG. 7 shows cell expansion in the peripheral blood of cynomolgus monkeys after a single dose of a fusion protein comprising CD8a-hzB7v15 and an attenuated IL-2.

FIG. 8A-8B show binding of CD8a-targeting sdAbs formatted as VHH-homodimeric Fc fusion proteins or as VHH-knob-in-hole Fc fusion proteins comprising an attenuated IL-2 mutant, as assessed by flow cytometry. FIG. 8A shows binding to HEK 293F cells transfected with full-length human CD8α (CD8a-FL). FIG. 8B shows binding to HEK 293F cells transfected with full-length human CD8b (CD8b-FL).

FIG. 9A-9B show binding of CD8a-targeting sdAbs formatted as VHH-homodimeric Fc fusion proteins or as VHH-knob-in-hole Fc fusion proteins comprising an attenuated IL-2 mutant, as assessed by flow cytometry. FIG. 9A shows binding to CD8 T cells within pan T cells enriched from human whole blood. FIG. 9B shows a lack of binding to CD4 T cells within pan T cells enriched from human whole blood.

FIG. 10A-10H show binding of CD8a-targeting sdAbs formatted as VHH-hIgG1-Fc fusion proteins as assessed by flow cytometry. FIGS. 10A-10B and 10E-10F show binding to CD8 T cells (FIGS. 10A and 10D) or CD4 T cells (FIGS. 10B and 10F) within pan T cells enriched from human whole blood (FIGS. 10A and 10B), or peripheral blood mononuclear cells (PBMC) (FIGS. 10E and 10F). FIGS. 10C-10D and 10F-10H show binding to CD8 T cells (FIGS. 10C and 10G) or CD4 T cells (FIGS. 10D and 10H) within peripheral blood mononuclear cells (PBMC) isolated from cynomolgus monkey whole blood.

FIG. 11A-11B show STAT5 signaling cell populations within the peripheral blood of human donors. Shown are the levels of phosphorylated STAT5 (pSTAT5) (FIG. 11A-11B) or the percentage of cells expressing pSTAT5 (FIG. 11C-11D) in CD8 T cells (FIG. 11A, 11C) or regulatory T cells (Tregs, FIG. 11B) or CD4 T cells (FIG. 11D) within pan T cells enriched from human whole blood. Cells were treated with a fusion protein comprising CD8a-hzB7v31 or CD8aB7v41, an Fc region, and a mutant, attenuated IL-2; a fusion protein comprising a CD8a-hzB7v31 and an Fc region (no IL-2); a fusion protein comprising a non-targeted VHH, an Fc region, and the attenuated IL-2; or wild type IL-2.

FIG. 12A-12C show expansion of CD8 T cells (FIGS. 12A and 12C) or CD4 T cells (FIG. 12B) within dissociated tumor cell preparations from human tumor samples (two head and neck or kidney cancer cases and one colon cancer case, FIGS. 12A and 12B) or PBMC from a healthy blood donor (FIG. 12C) treated ex vivo with a fusion protein comprising CD8a-hzB7v31, an Fc region, and a mutant, attenuated IL-2; a fusion protein comprising a CD8a-hzB7v31 and an Fc region (no IL-2); a fusion protein comprising a non-targeted VHH, an Fc region, and the attenuated IL-2; or wild type IL-2.

FIG. 13A-13B show the activity of a single dose (1 mg/kg) of a fusion protein comprising CD8a-hzB7v15, an Fc region, and a mutant, attenuated IL-2 in cynomolgus monkeys. FIG. 13A shows the expansion of certain PBMC subpopulations as the fold change, relative to baseline, of cell numbers seven days after dosing. FIG. 13B shows the percentages of Ki67+ cells within these subpopulations before dosing (baseline) and seven days after dosing.

FIG. 14A-14B show the cytotoxic activity of enriched, pre-stimulated CD8 T cells (FIG. 14A) or antibody-dependent cellular cytotoxicity (ADCC) of PBMC (FIG. 14B) towards A431 epidermoid carcinoma cells. Cells were treated with a fusion protein comprising CD8a-hzB7v31, an Fc region, and an attenuated IL-2 mutant; a fusion protein comprising a non-targeted VHH, an Fc region, and the attenuated IL-2 mutant; or wild type IL-2, as indicated. CD8 T cells or PBMC were added at different effector-to-target cell ratios (20:1, 10:1 or 5:1) as indicated. The EGFR-specific therapeutic antibody cetuximab was added to cell cultures in FIG. 14B.

### DETAILED DESCRIPTION

Embodiments provided herein relate to CD8-binding polypeptides and their use in various methods of treating, for example, cancer.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or CD8-related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. In some embodiments, a polypeptide is a “complex” of a first polypeptide and a second polypeptide.

The terms “CD8a” and “CD8” are used interchangeably herein to refer to any native, mature CD8 that results from processing of a CD8 precursor in a cell. The term includes CD8 from any vertebrate source, including mammals such as primates (e.g., humans and cynomolgus or rhesus monkeys) and rodents (e.g., mice and rats), unless otherwise indicated. The term also includes naturally-occurring variants of CD8, such as splice variants or allelic variants. A nonlimiting exemplary mature human CD8 amino acid sequence is shown, e.g., in NCBI Accession No. NP 001369627.1. See SEQ ID NO. 1.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a CD8 epitope is a sdAb or VHH-containing polypeptide that binds this epitope with greater affinity, avidity, more readily, and/or with greater duration than it binds to other CD8 epitopes or non-CD8 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “direct inhibition” and similar terms refers to an inhibition profile in which increasing antibody concentrations result in increasing inhibition. In some embodiments, after a certain concentration, maximal inhibition is reached and the inhibition profile plateaus. Maximal inhibition need not be 100% inhibition, but may be at least 50%, 55%, 60%, 65%, 70%, 75%, 80%, 85%, or 90%.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH1-VHH2-Fc or VHH1-VHH2-VHH3-Fc, wherein VHH1, VHH2, and VHH3 may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH1-Fc, VHH1-VHH2-Fc, and VHH1-VHH2-VHH3-Fc, wherein VHH1, VHH2, and VHH3 may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, the linker comprises: Gly-Gly-Gly-Gly (SEQ ID NO: 112), Gly-Gly-Ser-Gly-Gly-Ser (SEQ ID NO:113), and/or Gly-Gly-Ser-Ser-Gly-Ser (SEQ ID NO: 114). In some embodiments, when a VHH-containing polypeptide comprises an Fc, it forms a dimer. Thus, the structure VHH1-VHH2-Fc, if it forms a dimer, is considered to be tetravalent (i.e., the dimer has four VHH domains). Similarly, the structure VHH1-VHH2-VHH3-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3. In some embodiments, the CDRs are defined in accordance with the AbM definition.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4. In some embodiments, when an Fc region comprises a hinge, the hinge is of the same isotype as the Fc region. In some embodiments, an IgG4 hinge comprises a S228P stabilizing mutation.

An “acceptor human framework” as used herein is a framework comprising the amino acid sequence of a heavy chain variable domain (VH) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as T cell proliferation), and inducing or increasing expression of cytokines.

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells (Treg cells); macrophages; neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary CD8-Binding Polypeptides

Antagonist CD8-binding polypeptides are provided herein. In various embodiments, the CD8-binding polypeptides comprise at least one VHH domain that binds CD8. In some embodiments, a CD8-binding polypeptide provided herein comprises one, two, three, four, five, six, seven, or eight VHH domains that bind CD8. In some embodiments, a CD8-binding polypeptide provided herein comprises one, two, three, or four VHH domains that bind CD8. Such CD8-binding polypeptides may comprise one or more additional antigen-binding domains (e.g., VHH domains) that bind one or more target proteins other than CD8 and/or may comprise one or more additional polypeptide sequences, such as cytokine sequences.

In some embodiments, a CD8-binding polypeptide comprises at least one VHH domain that binds CD8 and an Fc region. In some embodiments, a CD8-binding polypeptide provided herein comprises one, two, three, or four VHH domains that bind CD8 and an Fc region. In some embodiments, an Fc region mediates dimerization of the CD8-binding polypeptide at physiological conditions such that a dimer is formed that doubles the number of CD8 binding sites. For example, a CD8-binding polypeptide comprising three VHH domains that bind CD8 and an Fc region is trivalent as a monomer, but at physiological conditions, the Fc region may mediate dimerization, such that the CD8-binding polypeptide exists as a hexavalent dimer under such conditions.

In some embodiments, a CD8-binding polypeptide comprises at least two VHH domains, wherein a first VHH domain binds a first epitope of CD8 and a second VHH domain binds a second epitope of CD8. When the CD8-binding polypeptide comprises a VHH domain that binds a first epitope of CD8 and a VHH domain that binds a second epitope of CD8, the CD8-binding polypeptide may be referred to as “biepitopic” or “bispecific.”

### CD8-Binding Polypeptides

In various embodiments, a VHH domain that binds CD8 comprises a CDR1 sequence selected from SEQ ID NOs: 3, 73, and 74, a CDR2 sequence selected from SEQ ID NOs: 4, 12, 14, 22, 27, 29, 31, 75, 76, 77, 78, 79, and 80, and a CDR3 selected from SEQ ID NOs: 5, 16, and 18. In various embodiments, a VHH domain that binds CD8 comprises CDR1, CDR2, and CDR3 sequences selected from: SEQ ID NOs: 3, 4, and 5; SEQ ID NOs: 3, 12, and 5; 3, 14, and 5; 3, 4, and 16; 3, 4, and 18; 3, 22, and 5; 3, 14, and 18; 3, 27, and 5; 3, 29, and 5; 3, 31, and 5; 73, 14, and 18; 74, 14, and 18; 3, 75, and 18; 3, 76, and 18; 3, 77, and 18; 3, 78, and 18; 3, 79, and 18; and 3, 80, and 18. In various embodiments, the VHH domain is humanized.

In some embodiments, a VHH domain that binds CD8 comprises an amino acid sequence that is at least 85%, at least 90%, at least 95%, at least 96%, at least 97%, at least 98%, at least 99% identical to an amino acid sequence selected from SEQ ID NOs: 2, 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, 99, and 100. In some embodiments, a VHH domain that binds CD8 comprises an amino acid sequence selected from SEQ ID NOs: 2, 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, 99, and 100. In some embodiments, a VHH domain that binds CD8 comprises an amino acid sequence selected from SEQ ID NOs: 2, 6, 7, 8, 9, 10, 11, 13, 15, 17, 19, 20, 21, 23, 24, 25, 26, 28 and 30, wherein residues XX are absent. In some embodiments, a VHH domain that binds CD8 comprises an amino acid sequence selected from SEQ ID NOs: 2, 6, 7, 8, 9, 10, 11, 13, 15, 17, 19, 20, 21, 23, 24, 25, 26, 28, 30, wherein residues XX are Gly-Gly. In some embodiments, a VHH domain that binds CD8 comprises an amino acid sequence selected from SEQ ID NOs: 2, 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, and 99, wherein the VHH domain comprises the mutation K117D, K117E, or K117R.

In some embodiments, a VHH domain that binds CD8 comprises a CDR1 sequence of SEQ ID NO: 3, a CDR2 sequence of SEQ ID NO: 14, and a CDR3 of SEQ ID NO: 18. In some embodiments, a VHH domain that binds CD8 comprises an amino acid sequence that is at least 85%, at least 90%, at least 95%, at least 96%, at least 97%, at least 98%, at least 99% identical to the amino acid sequence of SEQ ID NOs: 25. In some embodiments, a VHH domain that binds CD8 comprises the amino acid sequence of SEQ ID NO: 25. In some embodiments, a VHH domain that binds CD8 comprises a CDR1 sequence of SEQ ID NO: 3, a CDR2 sequence of SEQ ID NO: 78, and a CDR3 of SEQ ID NO: 18. In some embodiments, a VHH domain that binds CD8 comprises an amino acid sequence that is at least 85%, at least 90%, at least 95%, at least 96%, at least 97%, at least 98%, at least 99% identical to the amino acid sequence of SEQ ID NOs: 92 or 100. In some embodiments, a VHH domain that binds CD8 comprises the amino acid sequence of SEQ ID NO: 92. In some embodiments, a VHH domain that binds CD8 comprises the amino acid sequence of SEQ ID NO: 100.

In various embodiments, a CD8-binding polypeptide comprises one, two, three, or four VHH domains that bind CD8.

In some embodiments, a VHH domain that binds CD8 may be humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13:1619-1633, and are further described, for example, in Riechmann et al., (1988) Nature 332:323-329; Queen et al., (1989) Proc. Natl Acad. Sci. USA 86:10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272:10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a CD8-binding polypeptide is a human Fc region, or is derived from a human Fc region.

In some embodiments, an Fc region included in a CD8-binding polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling.

In some embodiments, the Fc region included in a CD8-binding polypeptide is derived from a human Fc region and comprises mutations M252Y and M428V, herein referred to as “Fc-YV”. In some embodiments, such mutations enhance binding to FcRn at the acidic pH of the endosome (near 6.5), while losing detectable binding at neutral pH (about 7.2), allowing for enhanced FcRn mediated recycling and extended half-life.

In some embodiments, the Fc region included in a CD8-binding polypeptide is derived from a human Fc region and comprises mutations designed for heterodimerization, herein referred to as “knob” and “hole”. In some embodiments, the “knob” Fc region comprises the mutation T366W. In some embodiments, the “hole” Fc region comprises mutations T366S, L368A, and Y407V. In some embodiments, Fc regions used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hold Fc region comprises the modification H435R or H435K (referred to as “hole-R” in some instances when the modification is H435R), while the knob Fc region does not. In some instances, the hole-R mutation improves purification of the heterodimer over homodimeric hole Fc regions that may be present.

In some embodiments, the Fc region included in a CD8-binding polypeptide is derived from a human Fc region and lacks the C-terminal lysine residue (ΔK447).

Nonlimiting exemplary Fc regions that may be used in a CD8-binding polypeptide include Fc regions comprising the amino acid sequences of SEQ ID NOs: 32-70, and 101-111. In some embodiments, a CD8-binding polypeptide includes an Fc region comprising an amino acid sequence selected from SEQ ID NOs: 33, 36-52, 68-70, and 101-111.

### Exemplary Activities of CD8-Binding Polypeptides

In various embodiments, the CD8-binding polypeptides provided herein stimulate CD8+ cells in vitro and/or in vivo. Stimulation or activity of CD8+ cells in vitro and/or in vivo may be determined, in some embodiments, using the methods provided in the Examples herein.

In some embodiments, the CD8-binding polypeptides provided herein comprise an immune cell activating cytokine or an antigen-binding domain that binds an antigen other than CD8 and stimulates CD8+ T cells. In some embodiments, the CD8+ stimulating activity of the immune cell activating cytokine or antigen-binding domain that binds an antigen other than CD8 is increased and/or more specifically targeted to cytotoxic T cells when fused to a CD8-binding VHH than when used alone. In some embodiments, toxicity of an immune cell activating cytokine or an antigen-binding domain that binds an antigen other than CD8 is reduced by specifically targeting it to CD8+ T cells.

In some embodiments, the CD8-binding polypeptides comprising an immune cell activating cytokine or an antigen-binding domain that binds an antigen other than CD8 provided herein increase T cell proliferation in vitro and/or in vivo.

In some embodiments, the CD8-binding polypeptides provided herein comprise a CD8-binding VHH provided herein and an immune cell activating cytokine. In some such embodiments, the immune cell activating cytokine is IL-2, IL-15, IL-7, IL-6, IL-12, IFNα, IFNβ, or IFNγ. In some such embodiments, the immune cell activating cytokine is a wild type immune cell activating cytokine. In some embodiments, the immune cell activating cytokine comprises mutations that attenuate the activity of the immune cell activating cytokine relative to the activity of the wild type cytokine. In some embodiments, the CD8-binding polypeptide comprising an immune cell activating cytokine stimulates CD8+ T cell activation and proliferation in vivo. In some embodiments, the CD8-binding polypeptide comprising an immune cell activating cytokine are used in a method of treating cancer.

The increase in proliferation of activated CD8 T cells may be determined by any method in the art, such as for example, the methods provided in the Examples herein. A nonlimiting exemplary assay is as follows. CD8 T cells may be isolated from one or more healthy human donors. The T cells are stained with CellTrace Violet (CTV) and activated with anti-CD3 antibody, contacted with a polypeptide provided herein, and then analyzed by FACS. Loss of CTV staining indicates proliferation. In some embodiments, an increase in CD8 T cell proliferation is determined as an average from a set of experiments or from pooled T cells, such as by measuring proliferation of CD8 T cells isolated from different healthy human donors. In some embodiments, an increase in CD8 T cell proliferation is determined as an average from experiments carried out using T cells from at least five or at least ten different healthy donors, or from a pool of T cells from at least five or at least ten different healthy donors.

In some embodiments, the CD8-binding polypeptides provided herein comprise a CD8-binding VHH and an antigen-binding domain that binds an antigen other than CD8. In some such embodiments, the antigen is Lag3, CTLA4, TGFBR1, TGFBR2, Fas, TNFR2, PD1, PDL1, or TIM3. In some embodiments, the antigen is 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, PIGF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3. In some embodiments, the CD8-binding polypeptide comprises a CD8-binding VHH and an antigen-binding domain that binds a tumor cell antigen.

### Polypeptide Expression and Production

Nucleic acid molecules comprising polynucleotides that encode a CD8-binding polypeptide are provided. In some embodiments, the nucleic acid molecule may also encode a leader sequence that directs secretion of the CD8-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the CD8-binding polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a CD8-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6R cells (Crucell); and NSO cells. In some embodiments, the CD8-binding polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a CD8-binding polypeptide described herein is provided. The CD8-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a CD8-binding polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the CD8-binding polypeptide is produced in a cell-free system. Nonlimiting exemplary cell-free systems are described, for example, in Sitaraman et al., Methods Mol. Biol. 498:229-44 (2009); Spirin, Trends Biotechnol. 22:538-45 (2004); Endo et al., Biotechnol. Adv. 21:695-713 (2003).

In some embodiments, CD8-binding polypeptides prepared by the methods described above are provided. In some embodiments, the CD8-binding polypeptide is prepared in a host cell. In some embodiments, the CD8-binding polypeptide is prepared in a cell-free system. In some embodiments, the CD8-binding polypeptide is purified. In some embodiments, a cell culture media comprising a CD8-binding polypeptide is provided.

In some embodiments, compositions comprising antibodies prepared by the methods described above are provided. In some embodiments, the composition comprises a CD8-binding polypeptide prepared in a host cell. In some embodiments, the composition comprises a CD8-binding polypeptide prepared in a cell-free system. In some embodiments, the composition comprises a purified CD8-binding polypeptide.

### Exemplary Methods of Treating Diseases Using CD8-Binding Polypeptides

In some embodiments, methods of treating disease in an individual comprising administering a CD8-binding polypeptide are provided. Such diseases include any disease that would benefit from increased proliferation and activation of T cells, such as CD8 T cells. In some embodiments, methods for treating cancer in an individual are provided. In some embodiments, a method of treating cancer comprises increasing proliferation and/or activation of CD8+ T cells by administering a CD8-binding polypeptide comprising a CD8-binding VHH and an immune cell activating cytokine or an antigen-binding domain that binds a tumor cell antigen other than CD8.

The method comprises administering to the individual an effective amount of a CD8-binding polypeptide provided herein. Such methods of treatment may be in humans or animals. In some embodiments, methods of treating humans are provided. Nonlimiting exemplary cancers that may be treated with CD8-binding polypeptides provided herein include basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

The CD8-binding polypeptides can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a CD8-binding polypeptides is administered to a subject one or more times. In some embodiments, an effective dose of a CD8-binding polypeptides is administered to the subject daily, semiweekly, weekly, every two weeks, once a month, etc. An effective dose of a CD8-binding polypeptides is administered to the subject at least once. In some embodiments, the effective dose of a CD8-binding polypeptides may be administered multiple times, including multiple times over the course of at least a month, at least six months, or at least a year.

In some embodiments, pharmaceutical compositions are administered in an amount effective for treating (including prophylaxis of) cancer and/or increasing T-cell proliferation. The therapeutically effective amount is typically dependent on the weight of the subject being treated, his or her physical or health condition, the extensiveness of the condition to be treated, or the age of the subject being treated. In general, antibodies may be administered in an amount in the range of about 0.05 mg/kg body weight to about 100 mg/kg body weight per dose.

In some embodiments, CD8-binding polypeptides can be administered in vivo by various routes, including, but not limited to, intravenous, intra-arterial, parenteral, intraperitoneal or subcutaneous. The appropriate formulation and route of administration may be selected according to the intended application.

In some embodiments, a therapeutic treatment using a CD8-binding polypeptide is achieved by increasing T-cell proliferation and/or activation, and/or by bringing CD8+ T cells in contact with cancer cells. In some embodiments, increasing T-cell proliferation and/or activation inhibits growth of cancer.

### Pharmaceutical Compositions

In some embodiments, compositions comprising CD8-binding polypeptides are provided in formulations with a wide variety of pharmaceutically acceptable carriers (see, for example, Gennaro, Remington: The Science and Practice of Pharmacy with Facts and Comparisons: Drug facts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprises a CD8-binding polypeptide at a concentration of at least 10 mg/mL.

### Combination Therapy

CD8-binding polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, a CD8-binding polypeptide provided herein is given concurrently with a second therapeutic agent, for example, a PD-1 or PD-L1 therapy. Examples of PD-1/PD-L1 therapy include nivolumab (BMS); pidilizumab (CureTech, CT-011), pembrolizumab (Merck); durvalumab (Medimmune/AstraZeneca); atezolizumab (Genentech/Roche); avelumab (Pfizer); AMP-224 (Amplimmune); BMS-936559; AMP-514 (Amplimmune); MDX-1105 (Merck); TSR-042 (Tesaro/AnaptysBio, ANB-011); STI-A1010 (Sorrento Therapeutics); STI-A1110 (Sorrento Therapeutics); and other agents that are directed against programmed death-1 (PD-1) or programmed death ligand 1 (PD-L1).

In some embodiments, a CD8-binding polypeptide provided herein is given concurrently with an immune stimulatory agent, for example, an agonist of a member of the Tumor Necrosis Factor Receptor Super Family (TNFRSF) or a member the B7 family. Nonlimiting examples of immune stimulatory TNFRSF members include OX40, GITR, 41BB, CD27, and HVEM. Nonlimiting examples of B7 family members include CD28 and ICOS. Thus, in some embodiments, a CD8-binding polypeptide provided herein is given concurrently with an agonist, such as an agonist antibody, of OX40, GITR, 41BB, CD27, HVEM, CD28, and/or ICOS.

In some embodiments, a CD8-binding polypeptide provided herein is given concurrently with CAR-T (chimeric antigen receptor T-cell) therapy, oncolytic virus therapy, cytokine therapy, and/or agents that target other checkpoint molecules, such as VISTA, gpNMB, B7H3, B7H4, HHLA2, CTLA4, TIGIT, etc.

### Nonlimiting Exemplary Methods of Diagnosis and Treatment

In some embodiments, the methods described herein are useful for evaluating a subject and/or a specimen from a subject (e.g. a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the evaluation may direct treatment (including treatment with the antibodies described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the antibodies are used as an adjuvant therapy in the treatment of a cancer. In some embodiments, the antibodies are used as the sole adjuvant therapy in the treatment of a cancer. In some embodiments, the antibodies described herein are withheld as an adjuvant therapy in the treatment of a cancer. For example, if a patient is unlikely to respond to an antibody described herein or will have a minimal response, treatment may not be administered in the interest of quality of life and to avoid unnecessary toxicity from ineffective chemotherapies. In such cases, palliative care may be used.

In some embodiments the molecules are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means an antibody is administered to cancer patients prior to surgery. Types of cancers for which neoadjuvant chemotherapy is commonly considered include, for example, breast, colorectal, ovarian, cervical, bladder, and lung. In some embodiments, the antibodies are used as a neoadjuvant therapy in the treatment of a cancer. In some embodiments, the use is prior to resection.

In some embodiments, the tumor microenvironment contemplated in the methods described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells; macrophages; neutrophils; and other immune cells located proximal to a tumor.

### Kits

Also provided are articles of manufacture and kits that include any of CD8-binding polypeptides as described herein, and suitable packaging. In some embodiments, the invention includes a kit with (i) a CD8-binding polypeptide, and (ii) instructions for using the kit to administer the CD8-binding polypeptide to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Development of CD8a-Binding VHH Domains

Single domain antibodies targeting human CD8α were generated via immunization of llamas with the extracellular domain of human CD8α fused to llama Fc (SEQ ID NO: 72). Following the development of specific anti-CD8α antibody titers, llama peripheral blood mononuclear cells (PBMC) were isolated from 500 mL of blood from the immunized animal and total mRNA was isolated using the Qiagen RNeasy Maxi Kit and subsequently converted to first strand cDNA using Thermo Superscript IV Reverse Transcriptase and oligo-dT priming. VHH sequences were specifically amplified via PCR using the cDNA as template and cloned into a yeast surface display vector as VHH-Fc-AGA2 fusion proteins. The Fc was a human IgG1 Fc (SEQ ID NO: 32) or, in some cases, a variant IgG1 Fc with reduced effector function (e.g., Fc xELL; SEQ ID NO: 33).

Yeast libraries displaying the VHH-Fc-AGA2 fusion proteins were enriched using recombinant forms of the CD8α ECD via magnetic bead isolation followed by fluorescence activated cell sorting (FACS). Sorted yeast were plated out and isolated colonies were picked into 96-well blocks and grown in media that switched the expression from surface displayed VHH-Fc to secretion into the media. Supernatants from the 96-well yeast secretion cultures were applied to 293F cells transiently transfected with CD8α (CD8α positive) or untransfected 293F cells (CD8α negative), washed, treated with fluorophore labelled anti-human IgG1 Fc secondary antibody, and analyzed by 96-well flow cytometry.

Nucleic acid sequences encoding VHHs that bound to CD8α positive cells and not to CD8α negative cells were cloned in-frame with a human Fc xELL encoding region into mammalian expression vectors, and expressed by transient transfection in HEK293 Freestyle cells (293F cells) or CHO cells using polyethylenimine. Supernatant was collected after 3-7 days, secreted recombinant protein was purified by protein A chromatography, and concentration was calculated from the absorbance at 280 nm and extinction coefficient.

One VHH domain that binds CD8α (clone B7) was humanized. Briefly, various humanized forms of B7 were made based on human heavy chain frameworks. Certain amino acids were back-mutated to the donor amino acid, and certain mutations were tested, for example, in the CDRs for their binding properties. The amino acid sequences of B7 and the various humanized forms are provided in the Table of Certain Sequences provided below. It will be noted that the sequences of B7 VHH (SEQ ID NO:2) and the humanized forms hzB7v1-hzB7v 18 (SEQ ID NOs: 6-30 may include an optional Gly-Gly (GG) linker at their C-terminus (represented by XX in the Table of Certain Sequences). In addition, it is provided that the lysine at residue 117 (K117) in any of the disclosed VHH domains may be substituted with an aspartate (K117D), a glutamate (K117E), or an arginine (K117R). The humanized VHH designated hzB7v41 (SEQ ID NO:100) comprises a K117R substitution (shown bolded and underlined in the Table of Certain Sequences).

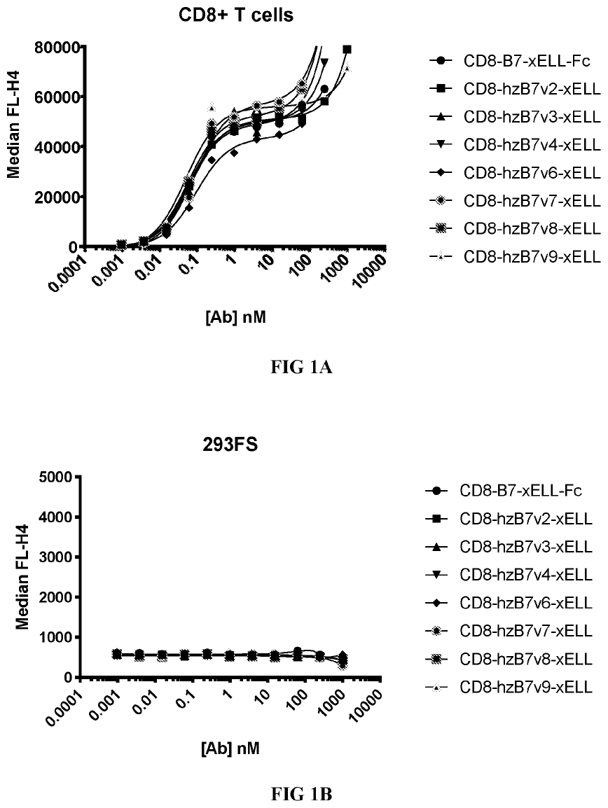

Binding of CD8a-binding polypeptides, formatted as CD8α VHH-hIgG1-Fc, was assessed by flow cytometry on isolated human CD8+ T cells. The isolated T cells were plated in a 96-well plate at 30,000 cells per well in FACS buffer (PBS, 1% BSA, 0.1% NaN₃, pH 7.4). Untransfected HEK293F cells were used as an CD8a-negative control and plated at 30,000 cells per well in a separate plate. Test polypeptides were then diluted to 2× the final concentration of 1000 nM and 3-, 4-, and 5-fold serial dilutions were made. FACS buffer with no polypeptide was used as a secondary antibody-only control. Polypeptide dilutions were added to an equal volume of cells, and assay plates were incubated for 30 minutes at 4° C. After washing twice with 150 μL of FACS buffer per well, the cells were resuspended in FACS buffer with fluorescently-labeled anti-human Fc antibody diluted 1:2000 to detect binding, and a fluorescently-labeled anti-CD4 antibody (clone OKT4) diluted 1:100 as a counter-stain. Assay plates were incubated at 4° C. for 20 minutes. After one additional wash with 150 μL of FACS buffer per well, bound polypeptide was detected by flow cytometry on CD4− cells. CD8α binding was measured on this population as median fluorescence at 647 nm. The data was plotted and analyzed using GraphPad Prism analysis software. Flow cytometric detection was performed on an Intellicyt iQue Plus. The resulting maximal binding (Bmax) values and binding affinities (K_{d}) are shown in Tables 2 and 3, and the binding curves are shown in FIGS. 1A-B and 2A-B. Higher Bmax values generally indicate slower off-rates, which lower Bmax values likely indicate faster off-rates.

TABLE 2

| Fusion Protein | Bmax (MFI) | K_{d} (nM) | SEQ ID NOs. |
|---|---|---|---|
| B7-xELL-Fc | 50903 | 0.06072 | 2, 33 |
| hzB7v2-xELL-Fc | 51439 | 0.06946 | 7, 33 |
| hzB7v3-xELL-Fc | 50103 | 0.05792 | 8, 33 |
| hzB7v4-xELL-Fc | 50839 | 0.05671 | 9, 33 |
| hzB7v6-xELL-Fc | 43658 | 0.09356 | 11, 33 |
| hzB7v7-xELL-Fc | 58151 | 0.08346 | 13, 33 |
| hzB7v8-xELL-Fc | 53344 | 0.05797 | 15, 33 |
| hzB7v9-xELL-Fc | 57324 | 0.05084 | 17, 33 |

TABLE 3

| Fusion Protein | Bmax (MFI) | K_{d} (nM) | SEQ ID NOs. |
|---|---|---|---|
| B7-xELL-Fc | 39806 | 0.06663 | 2, 33 |
| hzB7v2-xELL-Fc | 38832 | 0.05951 | 7, 33 |
| hzB7v10-xELL-Fc | 26561 | 0.1065 | 19, 33 |
| hzB7v11-xELL-Fc | 41041 | 0.06089 | 20, 33 |
| hzB7v12-xELL-Fc | 29460 | 0.05847 | 21, 33 |
| hzB7v13-xELL-Fc | 31494 | 0.05805 | 23, 33 |
| hzB7v14-xELL-Fc | 36208 | 0.06564 | 24, 33 |
| hzB7v15-xELL-Fc | 39345 | 0.09653 | 25, 33 |

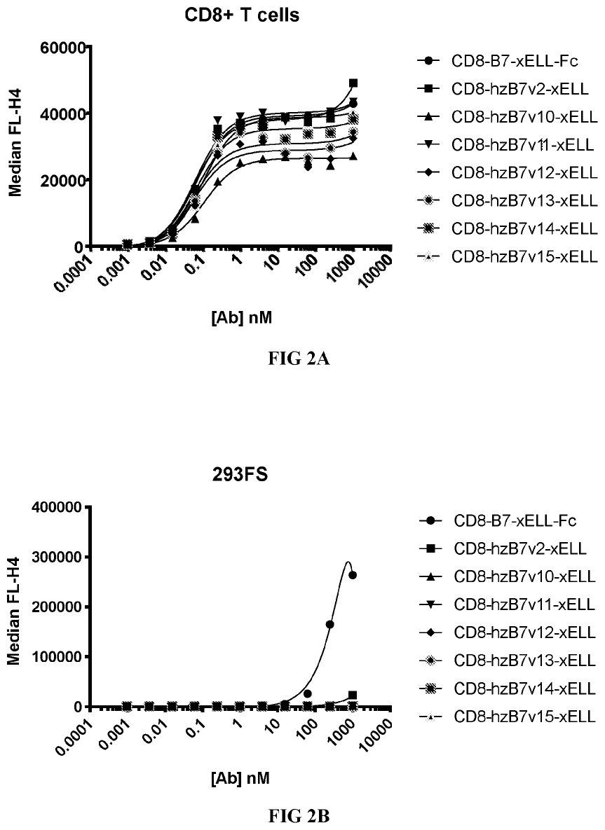

As shown in FIGS. 1A and 2A and the tables above, the tested CD8-binding polypeptides bound human CD8+ T cells with affinities below 0.2 nM, and in most instances, below 0.1 nM. As shown in FIGS. 1B and 2B, all of the tested polypeptides except parental B7-xELL-Fc exhibited no significant binding to 293 control cells, and B7-xELL-Fc bound the control cells with more than 2,000-fold reduced affinity compared to binding to CD8+ T cells. These results demonstrated that the CD8a-binding polypeptides specifically bound CD8.

### Example 2: Binding of CD8-Binding Polypeptides to Human and Cynomolgus Monkey CD8

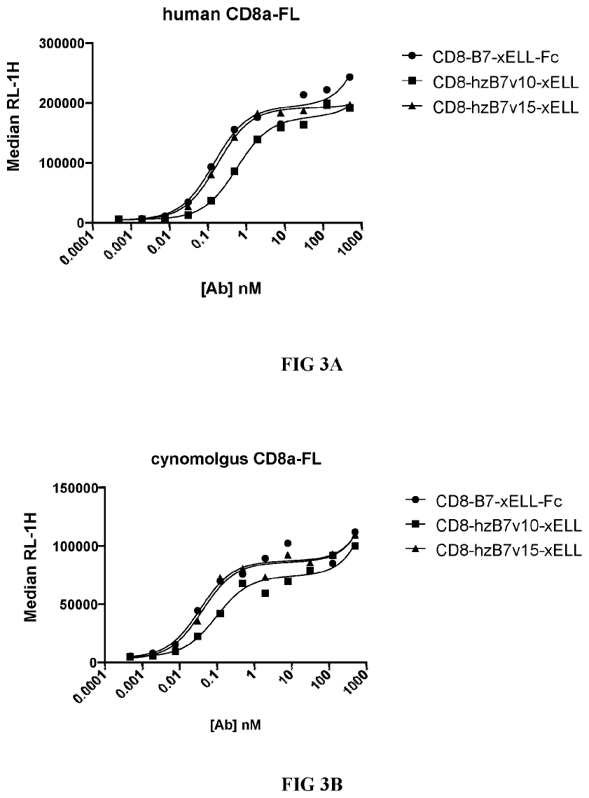

Binding of parental and two of the humanized CD8a-binding polypeptides described above was assessed by flow cytometry on transfected HEK293F cells. The HEK293F cells were transiently transfected with a plasmid encoding full length human or cynomolgus monkey CD8α followed by an IRES and GFP. The transfected cells were plated in a 96-well plate at 30,000 cells per well in FACS buffer (PBS, 1% BSA, 0.1% NaN₃, pH 7.4). Test polypeptides were then diluted to 2× the final concentration of 500 nM and 3-, 4-, and 5-fold serial dilutions were made. FACS buffer with no polypeptide was used as a secondary antibody-only control. Test polypeptides were added to an equal volume of cells, and assay plates were incubated for 30 minutes at 4° C. After washing twice with 150 μL of FACS buffer per well, the cells were resuspended in FACS buffer with fluorescently-labeled anti-human Fc antibody diluted 1:2000. Assay plates were incubated at 4° C. for 20 minutes. After one additional wash with 150 μL of FACS buffer, bound polypeptide was detected by flow cytometry. Flow cytometric detection was performed on an Intellicyt iQue Plus. CD8a-expressing transfected cells were gated as GFP positive, and polypeptide binding was measured as median fluorescence at 647 nm. The data was plotted and analyzed using GraphPad Prism analysis software. The results are shown in the Tables 4 and 5 and in FIGS. 3A-B.

TABLE 4
Binding on human CD8a transfected HEK293F cells

| Fusion Protein | Bmax (MFI) | K_{d} (nM) | SEQ ID NOs. |
|---|---|---|---|
| B7-xELL-Fc | 189563 | 0.1448 | 2, 33 |
| hzB7v10-xELL-Fc | 171061 | 0.5654 | 19, 33 |
| hzB7v15-xELL-Fc | 187850 | 0.1784 | 25, 33 |

TABLE 5
Binding on cynomolgus monkey CD8a transfected HEK293F cells

| Fusion Protein | Bmax (MFI) | K_{d} (nM) | SEQ ID NOs. |
|---|---|---|---|
| B7-xELL-Fc | 82948 | 0.03533 | 2, 33 |
| hzB7v10-xELL-Fc | 69358 | 0.09725 | 19, 33 |
| hzB7v15-xELL-Fc | 82954 | 0.04056 | 25, 33 |

As shown in FIG. 3A and Table 4, the tested CD8-binding polypeptides bound transfected HEK293F cells expressing human CD8α with affinities below 0.6 nM. As shown in FIG. 3B and Table 5, the tested CD8-binding polypeptides bound HEK293F cells expressing cynomolgus monkey CD8α with affinities below 0.1 nM.

### Example 3: CD8-Binding Polypeptides Bind to Human and Cynomolgus Monkey Immune Cells

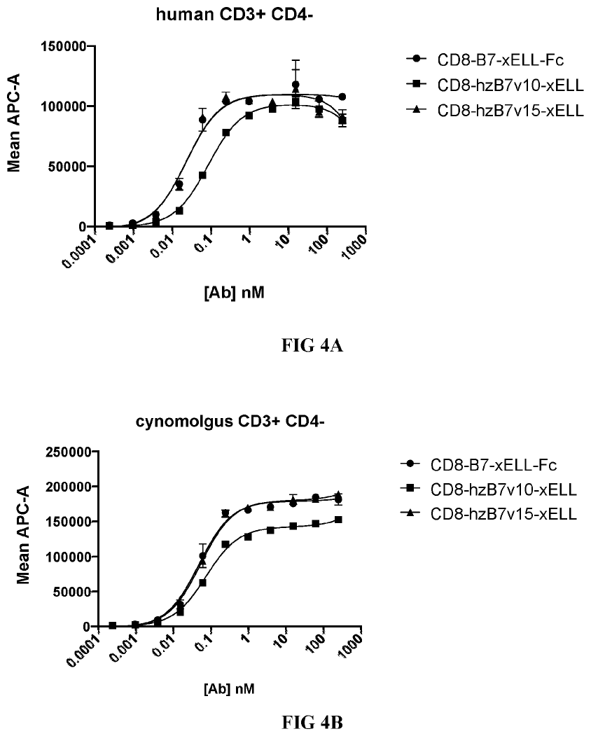

Binding of parental and two humanized CD8a-binding polypeptides described above was assessed by flow cytometry on isolated human T cells and on isolated cynomolgus monkey PBMC. The isolated cells were plated in a 96-well plate at 200,000 cells per well for cynomolgus monkey PBMC, and 50,000 cells per well for human T cells in FACS buffer (PBS, 1% BSA, 0.1% NaN₃, pH 7.4). Test polypeptides were then diluted to 2× the final concentration of 250 nM and a 4-fold serial dilution was made. FACS buffer alone was used as a secondary antibody-only control. Polypeptide dilutions were added to an equal volume of cells, and assay plates were incubated for 30 minutes at 4° C. After washing twice with 150 μL of FACS buffer per well, the cells were resuspended in FACS buffer with fluorescently-labeled anti-human antibody diluted 1:1000 to detect CD8α binding, and fluorescently labeled anti-CD3 antibody (clone SP34.2) diluted 1:40, and anti-CD4 antibody (clone OKT4) diluted 1:100. Assay plates were incubated at 4° C. for 20 minutes. After one additional wash with 150 μL of FACS buffer, CD8α binding was detected by flow cytometry on CD3+CD4− cells. Binding was measured on these cell populations as mean fluorescence at 647 nm. The data was plotted and analyzed using GraphPad Prism analysis software. Flow cytometric detection was performed on an ACEA Biosciences Novocyte-Quanteon Flow Cytometer. The results are shown in Table 6 and 7 and in FIGS. 4A-B.

TABLE 6
Binding on human CD3+ CD4− T cells

| Fusion Protein | Bmax (MFI) | K_{d} (nM) | SEQ ID NOs. |
|---|---|---|---|
| B7-xELL-Fc | 111947 | 0.02335 | 2, 33 |
| hzB7v10-xELL-Fc | 102521 | 0.08498 | 19, 33 |
| hzB7v15-xELL-Fc | 112777 | 0.02296 | 25, 33 |

TABLE 7
Binding on cynomolgus monkey CD3+ CD4− cells

| Fusion Protein | Bmax (MFI) | K_{d} (nM) | SEQ ID NOs. |
|---|---|---|---|
| B7-xELL-Fc | 180823 | 0.04832 | 2, 33 |
| hzB7v10-xELL-Fc | 142617 | 0.07370 | 19, 33 |
| hzB7v15-xELL-Fc | 181851 | 0.05399 | 25, 33 |

As shown in FIG. 4A and Table 6, the tested CD8-binding polypeptides bound human CD3+CD4− T cells with affinities below 0.1 nM. As shown in FIG. 4B and Table 7, the tested CD8-binding polypeptides bound cynomolgus CD3+CD4− cells with affinities below 0.08 nM.

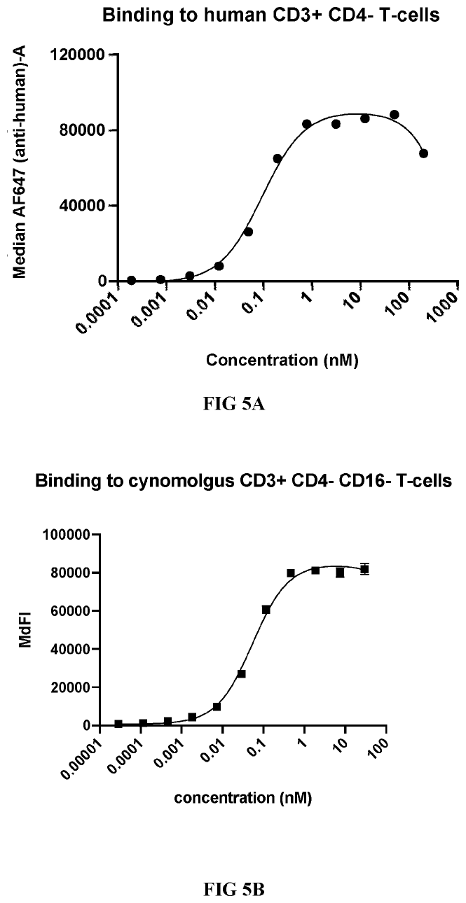

Binding of CD8a-binding polypeptide hzB7v15-xELL-Fc was assessed by flow cytometry on human leukopak T cells and on cynomolgus monkey PBMC. The leukopak T cells were thawed with CTL anti-aggregate wash thawing solution and plated in a 96-well U-bottom assay plate. Cells were centrifuged at 400×g for 5 minutes, and the supernatant discarded. hzB7v15-xELL-Fc was serially diluted 1:3 across 10 wells from an initial concentration of 200 nM. FACS buffer was used as a non-binding control, and the plate was incubated at 4° C. for 30 minutes. The assay plate was centrifuged at 400×g for 5 minutes and the supernatant discarded. Cells were washed once and resuspended in the staining panel (anti-CD3 antibody clone OKT3-BV605 (1:200), anti-CD4 antibody clone OKT4-BV785 (1:200), and fluorescently-labeled anti-human Fc antibody (1:500)) for 30 minutes at 4° C. The assay plate was centrifuged at 400×g for 5 minutes and the supernatant discarded. Cells were washed with 150 μL of FACS buffer, and re-suspended with 70 uL FACS buffer for readout on a Novocyte flow cytometer. The results are shown in Table 8 below and in FIG. 5A.

Cynomolgus monkey PBMC were thawed with CTL anti-aggregate wash thawing solution and plated in a 96-well U-bottom assay plate at 500,000 cells per well. Cells were centrifuged at 400×g for 5 minutes and the supernatant discarded. Alexa Fluor 647 chemically labeled hzB7v15-xELL-Fc (AF647-hzB7v15-xELL-Fc) was serially diluted 1:3 across 10 wells from an initial assay concentration of 30 nM. FACS buffer was used as a non-binding control, and the plate was incubated at 4° C. for 20 minutes. The assay plate was centrifuged at 400×g for 5 minutes and the supernatant discarded. Cells were washed with 150 μL of PBS buffer, and re-suspended with 40 uL FACS buffer, 10 μL of BV staining buffer (Brilliant Stain Buffer Plus; BD Biosciences), and 50 μL of a mixture of antibodies (anti-CD3 antibody clone SP34-BV421 (1:25), anti-CD4 antibody clone OKT4-BV785 (1:100), and anti-CD16 antibody clone 3G8-PE (1:100)) in FACS buffer were added to the cells. Cells were stained for 20 minutes at 4° C. The assay plate was centrifuged at 400×g for 5 minutes and the supernatant discarded. Cells were washed with 150 μL of FACS buffer, and re-suspended with 70 uL FACS buffer for readout on a Novocyte flow cytometer. The results are shown in Table 9 below and in FIG. 5B.

TABLE 8
Binding on human CD3+ CD4− T cells

| Fusion Protein | K_{d} (nM) | SEQ ID NOs. |
|---|---|---|
| hzB7v15-xELL-Fc | 0.09465 | 25, 33 |

TABLE 9
Binding on cynomolgus monkey CD3+ CD4− CD16− cells

| Fusion Protein | K_{d} (nM) | SEQ ID NOs. |
|---|---|---|
| hzB7v15-xELL-Fc | 0.05412 | 25, 33 |

As shown in FIG. 5A and Table 8, hzB7v15-xELL-Fc bound human CD3+CD4− T cells with an affinity below 0.1 nM. As shown in FIG. 5B and Table 9, hzB7v15-xELL-Fcbound cynomolgus monkey CD3+CD4−CD16-cells with an affinity below 0.06 nM.

### Example 4: CD8a-Targeting of Attenuated IL-2 Restores Activity

CD8a-targeted IL-2 activity of polypeptides comprising CD8a-binding VHH hzB7v15 or VHH hzB7v31 domain, an Fc region, and an attenuated IL-2 fused to the C-terminus of the Fc region was assessed in IL-2 reporter cells. The fusion proteins were dimeric, comprising a VHH hzB7v15 or VHH hzB7v31 domain fused to a knob Fc region and an attenuated IL-2 and a VHH hzB7v15 or VHH hzB7v31 domain fused to a hole Fc region. Thus, the dimeric fusion protein comprised two CD8α binding VHH domain, two Fc regions, and one attenuated IL-2. HEK-Blue IL2 reporter cells or CD8a-expressing HEK-Blue IL2 reporter cells were detached, transferred to a 50 mL conical tube, pelleted at 400×g for 5 minutes, and resuspended in fresh, pre-warmed assay media (DMEM+4.5 g/L glucose, 2 mM L-glutamine+10% heat-inactivated FBS+100 U/mL penicillin+100 μg/mL streptomycin+100 μg/mL normocin) at a density of 0.5×10\hat{ }6 cells/ml. A polypeptide dilution series was prepared at 2× the final concentration in assay media, and 100 μL was added per well. 50,000 cells in 100 μL were added to each well in a flat-bottom 96-well tissue culture treated plate. Plates were incubated at 37° C. in a CO₂ incubator for 20 hours. Quanti-Blue solution was prepared following the manufacturer's instructions (resuspend in water and warm to 37° C. in water bath for 30 minutes). Assay plates were spun down at 400×g for 5 minutes. 100 μL of supernatant was transferred to a new flat-bottom 96-well tissue culture plate, and 100 μL/well of Quanti-Blue solution was added and incubated at 37° C. in a 5% CO₂ incubator for 1-2 hours. Absorbance was read at 650 nm on an EMax plate reader.

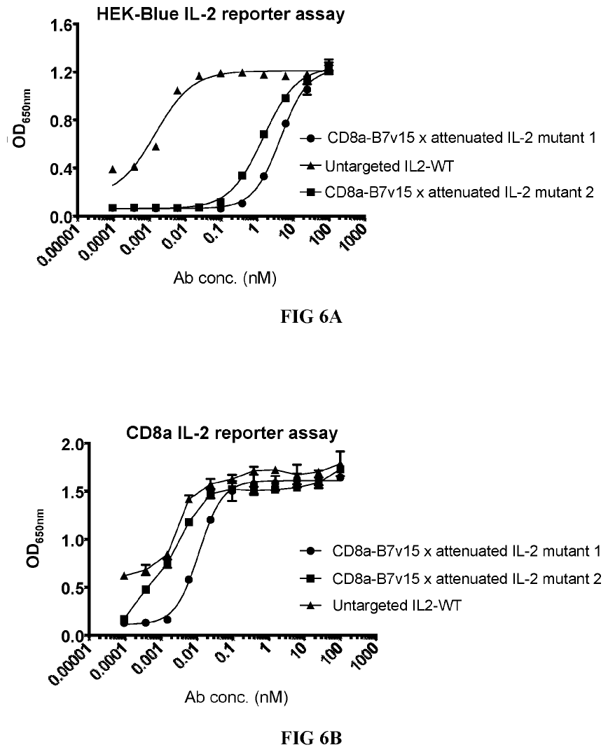

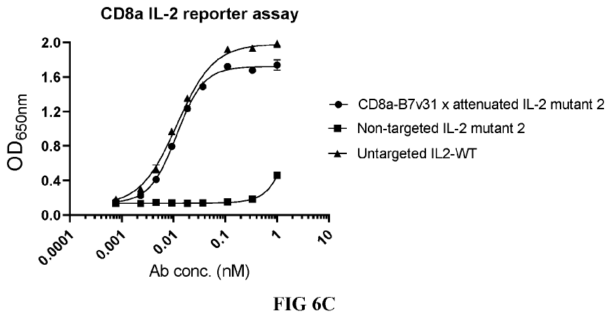

As shown in FIG. 6A, the CD8a-targeted polypeptides comprising an attenuated IL-2 exhibited significantly less activity than a polypeptide comprising a non-targeted VHH domain and wild type IL-2 on cells that do not express CD8a. As shown in FIGS. 6B and 6C, in cells expressing CD8a, the polypeptides comprising a CD8a-binding VHH and an attenuated IL-2 exhibited robust IL-2 activity, similar to that of a polypeptide comprising a non-targeted VHH domain and wild type IL-2. A polypeptide comprising a non-targeted VHH domain and the attenuated IL-2 exhibited significantly less activity on the CD8a-expressing reporter cells. These results show that IL-2 activity can be specifically targeted to CD8a-expressing cells within a broad concentration range, that in this reporter assay falls approximately between 0.01 to 1 nM.

### Example 5: T-Cell Proliferation Induced by Polypeptides Comprising a CD8a-Binding VHH and an Attenuated IL-2

The effects on CD8+ T cell expansion of a fusion protein comprising an attenuated IL-2 fused to the C-terminus of CD8a-binding VHH hzB7v15 were tested in non-human primates. Cynomolgus monkeys were administered an intravenous bolus injection of the fusion protein at 0.3 mg/kg. Whole blood samples were collected from the study animals before and 7 days after fusion protein administration. Peripheral blood mononuclear cells (PBMC) from each time point were isolated using density centrifugation in Lymphoprep™ and cells were stained with fluorescently-labeled cell type-specific antibody combinations. T cells were classified as CD3+ cells expressing CD4 or CD8α that did not express the B cell marker CD20. Regulatory T cells (“Tregs”) were defined as CD4+ T cells that also expressed CD25 and had reduced levels of CD127. CD4+ conventional T cells (“CD4+ Tcon”) were defined as CD4+ T cells that did not express CD25 and had normal levels of CD127. NK cells were defined as non-T and non-B cells expressing NKG2A. The population staining positive for CD20 was classified as B cells. Absolute cell counts of each PBMC subpopulation were determined using flow cytometry and fold-expansion was calculated by dividing the absolute cell count 7 days post dose by the baseline count pre-dose.

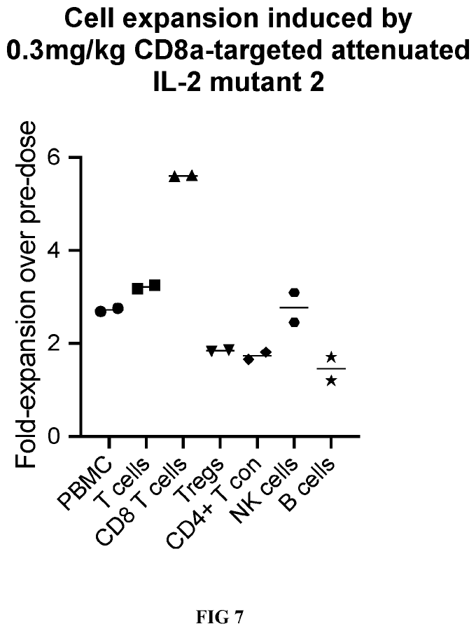

As shown in FIG. 7, a single dose of the CD8-targeted attenuated IL-2 at 0.3 mg/kg resulted in a 5.6-fold expansion of CD8+ T cells and a 2.8-fold expansion of NK cells, while not significantly affecting CD8-cell populations, including Tregs, CD4+ conventional T cells, and B cells. The higher numbers of CD8+ T cells also led to a 3.2-fold increase in overall T cells and total PBMC numbers were increased by 2.7-fold over the pre-dose cell counts. These data show that CD8a-targeted attenuated IL-2 specifically induced cell proliferation of CD8+ cell populations in vivo.

### Example 6: Binding of CD8a-Binding Polypeptides to Human CD8 Chains Expressed on 293F Cells

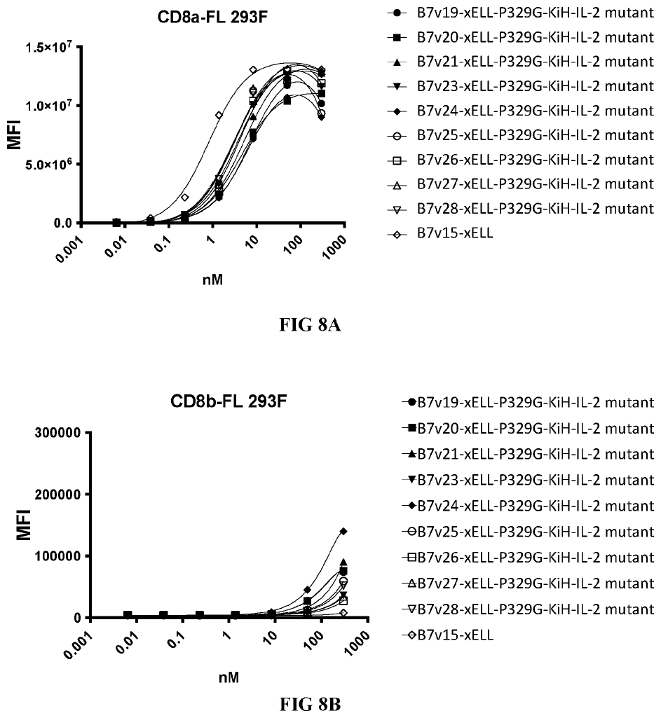

Binding of polypeptides comprising a humanized CD8a-binding VHH domain, an Fc region, and, in certain polypeptides, a mutated, attenuated IL-2 fused to the C-terminus of the Fc region, was assessed by flow cytometry on HEK293F cells transiently transfected with a plasmid encoding human CD8α or CD8b chains. Complexes or polypeptides labeled “KiH” comprise knob-in-hole heterodimeric Fc regions in which the indicated CD8a-binding VHH domain is fused to the N-terminus of each Fc region, and the mutant IL-2 is fused to the C-terminus of only the “knob” Fc region. Complexes or polypeptides that are not labeled “KiH” form homodimers under physiologic conditions. The transfected cells were plated in a 96-well plate at 50,000 cells per well in FACS buffer (PBS, 1% BSA, 0.1% NaN₃, pH 7.4). Test polypeptides were then diluted to 2× the final concentration of 500 nM, and a 6-fold serial dilution was made. FACS buffer with no polypeptide was used as a secondary antibody-only control. Test polypeptides were added to an equal volume of cells, and assay plates were incubated for 30 minutes at 4° C. After washing twice with 150 μL of FACS buffer per well, the cells were resuspended in FACS buffer with a fluorescently-labeled anti-human Fc antibody diluted 1:1000 to detect CD8 binding. Assay plates were incubated at 4° C. for 30 minutes. After one additional wash with 150 μL of FACS buffer, polypeptide bound to CD8 was detected by flow cytometry on cells positive for the transfection marker citrine. Binding was measured on these cell populations as mean fluorescence intensity (MFI) at 647 nm. Flow cytometric detection was performed on an IntelliCyt iQue Screener Plus. The data were plotted and analyzed using GraphPad Prism analysis software. The results are shown in Table 10 and in FIGS. 8A-B.

TABLE 10
Binding to human CD8a expressed on transfected HEK293F cells

| Fusion Protein | Bmax (MFI) | K_{d} (nM) | SEQ ID NOs. |
|---|---|---|---|
| hzB7v19-xELL- | 14162685 | 7.495 | 81, 68; and 81, 69, |
| P329G-KiH Fc-IL-2 | | | and mutant IL-2 |
| mutant | | | |
| hzB7v20-xELL- | 11553318 | 4.482 | 82, 68; and 82, 69, |
| P329G-KiH Fc-IL-2 | | | and mutant IL-2 |
| mutant | | | |
| hzB7v21-xELL- | 14220676 | 5.234 | 83, 68; and 83, 69, |
| P329G-KiH Fc-IL-2 | | | and mutant IL-2 |
| mutant | | | |
| hzB7v23-xELL- | 14341168 | 3.897 | 84, 68; and 84, 69, |
| P329G-KiH Fc-IL-2 | | | and mutant IL-2 |
| mutant | | | |
| hzB7v24-xELL- | 12760589 | 6.543 | 85, 68; and 85, 69, |
| P329G-KiH Fc-IL-2 | | | and mutant IL-2 |
| mutant | | | |
| hzB7v25-xELL- | 14542422 | 3.279 | 86, 68; and 86, 69, |
| P329G-KiH Fc-IL-2 | | | and mutant IL-2 |
| mutant | | | |
| hzB7v26-xELL- | 15247397 | 4.546 | 87, 68; and 87, 69, |
| P329G-KiH Fc-IL-2 | | | and mutant IL-2 |
| mutant | | | |
| hzB7v27-xELL- | 14456494 | 3.179 | 88, 68; and 88, 69, |
| P329G-KiH Fc-IL-2 | | | and mutant IL-2 |
| mutant | | | |
| hzB7v28-xELL- | 13748843 | 2.838 | 89, 68; and 89, 69, |
| P329G-KiH Fc-IL-2 | | | and mutant IL-2 |
| mutant | | | |
| hzB7v15-xELL Fc | 14220016 | 0.8207 | 25, 33 |

As shown in FIG. 8A and Table 10, the tested CD8a-binding polypeptides bound human CD8α with affinities in the low nanomolar range. FIG. 8B shows that the polypeptides bound to human CD8b with low to negligible affinity.

### Example 7: Binding of CD8a-Binding Polypeptides to T Cells

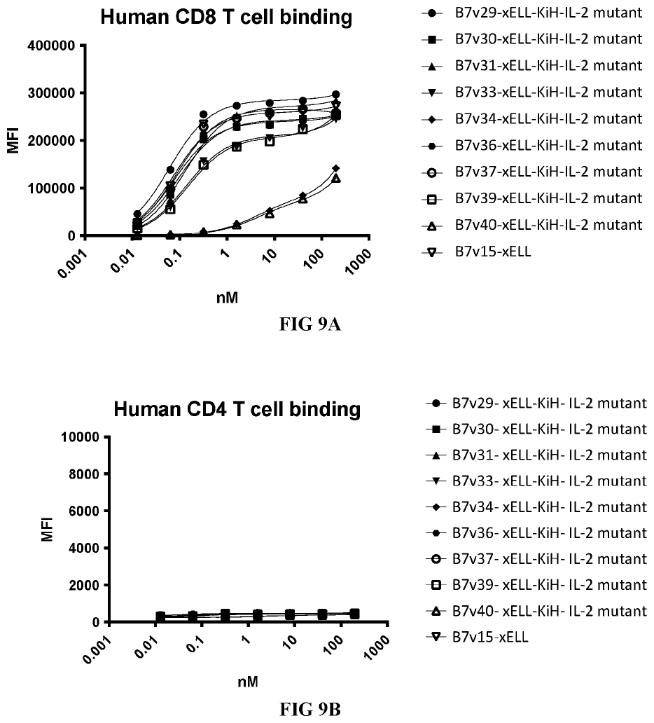

Binding of polypeptides comprising a humanized CD8a-binding VHH domain, an Fc region, and, in certain polypeptides, a mutated, attenuated IL-2 fused to the C-terminus of the Fc region, was assessed by flow cytometry on isolated human T cells. Complexes or polypeptides labeled “KiH” comprise knob-in-hole heterodimeric Fc regions in which the indicated CD8a-binding VHH domain is fused to the N-terminus of each Fc region, and the mutant IL-2 is fused to the C-terminus of only the “knob” Fc region. Complexes or polypeptides that are not labeled “KiH” form homodimers under physiologic conditions. The isolated cells were plated in a 96-well plate at 50,000 cells per well in FACS buffer (PBS, 1% BSA, 0.1% NaN₃, pH 7.4). Test polypeptides were then diluted to 2× the final concentration of 200 nM, and a 5-fold serial dilution was made. FACS buffer with no polypeptide was used as a secondary antibody-only control. Test polypeptides were added to an equal volume of cells, and assay plates were incubated for 30 minutes at 4° C. After washing twice with 150 μL of FACS buffer per well, the cells were resuspended in FACS buffer with a fluorescently-labeled anti-human IgG antibody diluted 1:1000 to detect CD8α binding and fluorescently labeled anti-CD4 antibody (clone OKT4, 1:200). Propidium iodide (PI) was added at 1:2000 to distinguish live cells from dead cells. Assay plates were incubated at 4° C. for 30 minutes. After one additional wash with 150 μL of FACS buffer, polypeptide bound to CD8α was detected by flow cytometry on PI-CD4− and on PI-CD4+ cells. Binding was measured on these cell populations as mean fluorescence intensity (MFI) at 647 nm. Flow cytometric detection was performed on an ACEA Biosciences Novocyte-Quanteon Flow Cytometer. The data were plotted and analyzed using GraphPad Prism analysis software. The results are shown in Table 11 and in FIGS. 9A-B.

TABLE 11
Binding to human CD8a expressed on transfected HEK293F cells

| Fusion Protein | Bmax (MFI) | K_{d} (nM) | SEQ ID NOs. |
|---|---|---|---|
| hzB7v29-xELL-KiH | 287960 | 0.0606 | 90, 48; and 90, 70, |
| Fc-IL-2 mutant | | | and mutant IL-2 |
| hzB7v30-xELL-KiH | 246762 | 0.09433 | 91, 48; and 91, 70, |
| Fc-IL-2 mutant | | | and mutant IL-2 |
| hzB7v31-xELL-KiH | 276473 | 0.1288 | 92, 48; and 92, 70, |
| Fc-IL-2 mutant | | | and mutant IL-2 |
| hzB7v33-xELL-KiH | 212072 | 0.1392 | 93, 48; and 93, 70, |
| Fc-IL-2 mutant | | | and mutant IL-2 |
| hzB7v34-xELL-KiH | 76068 | 4.049 | 94, 48; and 94, 70, |
| Fc-IL-2 mutant | | | and mutant IL-2 |
| hzB7v36-xELL-KiH | 245259 | 0.07723 | 95, 48; and 95, 70, |
| Fc-IL-2 mutant | | | and mutant IL-2 |
| hzB7v37-xELL-KiH | 272085 | 0.08843 | 96, 48; and 96, 70, |
| Fc-IL-2 mutant | | | and mutant IL-2 |
| hzB7v39-xELL-KiH | 209090 | 0.1484 | 97, 48; and 97, 70, |
| Fc-IL-2 mutant | | | and mutant IL-2 |
| hzB7v40-xELL-KiH | 71704 | 4.434 | 98, 48; and 98, 70, |
| Fc-IL-2 mutant | | | and mutant IL-2 |
| hzB7v15-xELL Fc | 266083 | 0.07576 | 25, 33 |

As shown in FIG. 9A and Table 11, the tested CD8a-binding polypeptides bound human CD8 T cells with affinities in the low nanomolar to sub-nanomolar range. FIG. 9B shows that the polypeptides did not bind to human CD4 T cells.

### Example 8: Binding of CD8a-Binding Polypeptides to Human and Cynomolgus Monkey CD8α

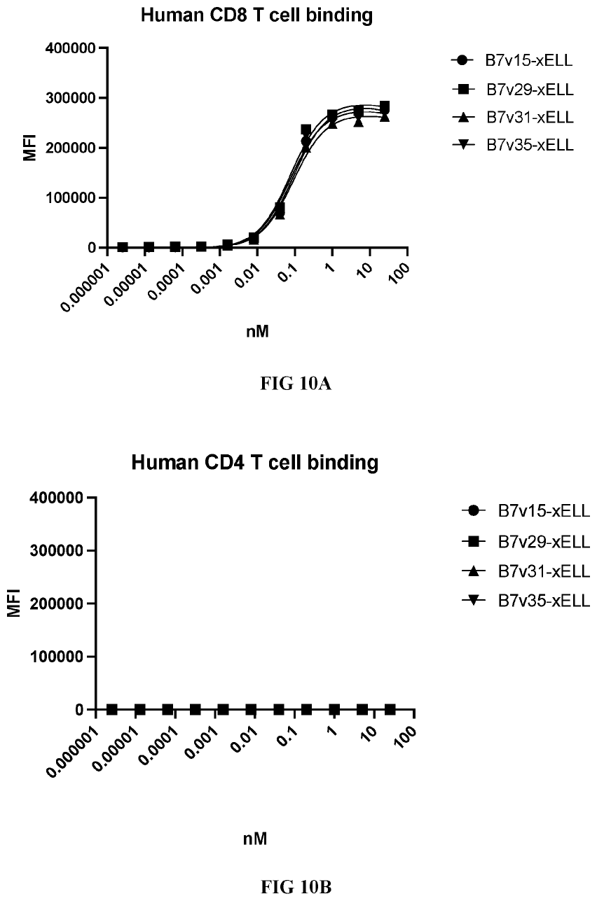

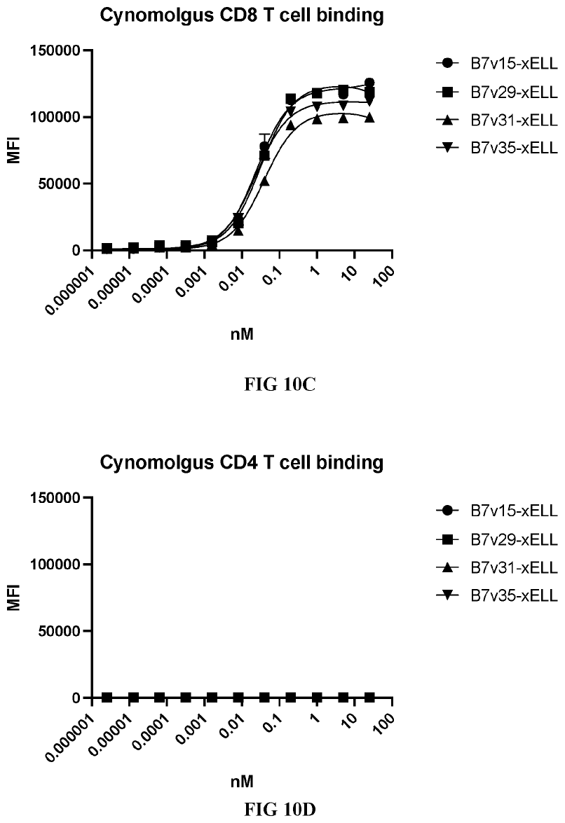

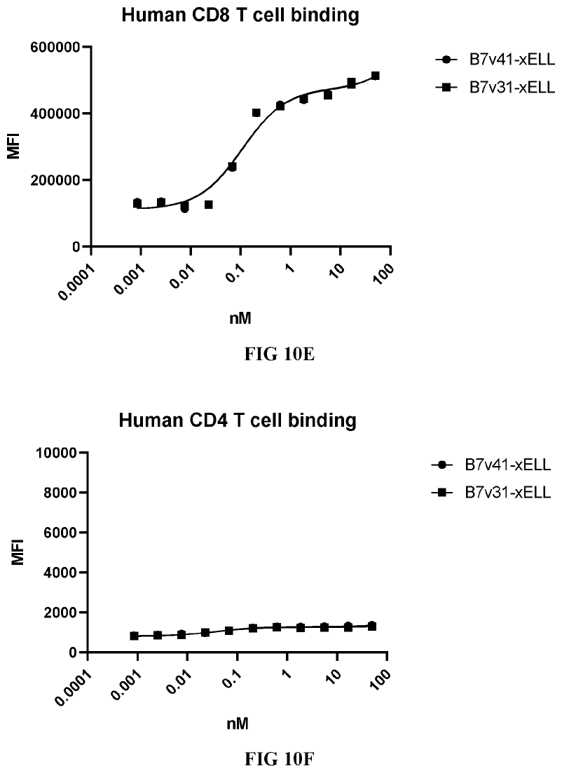

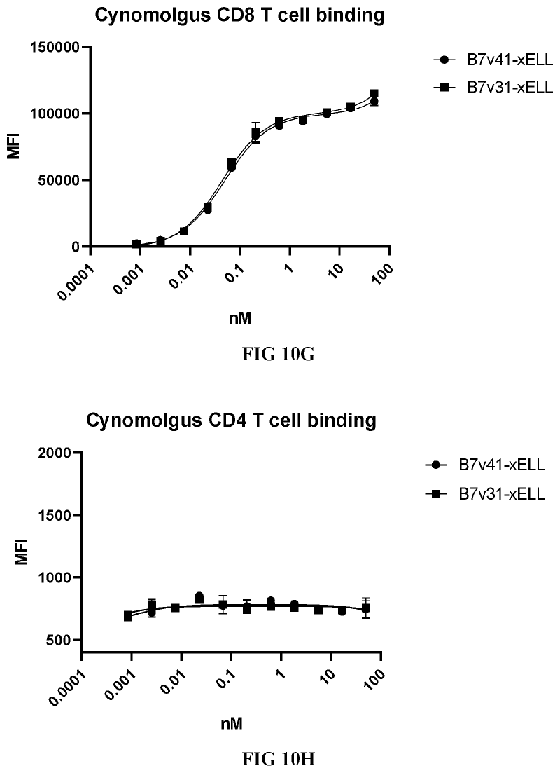

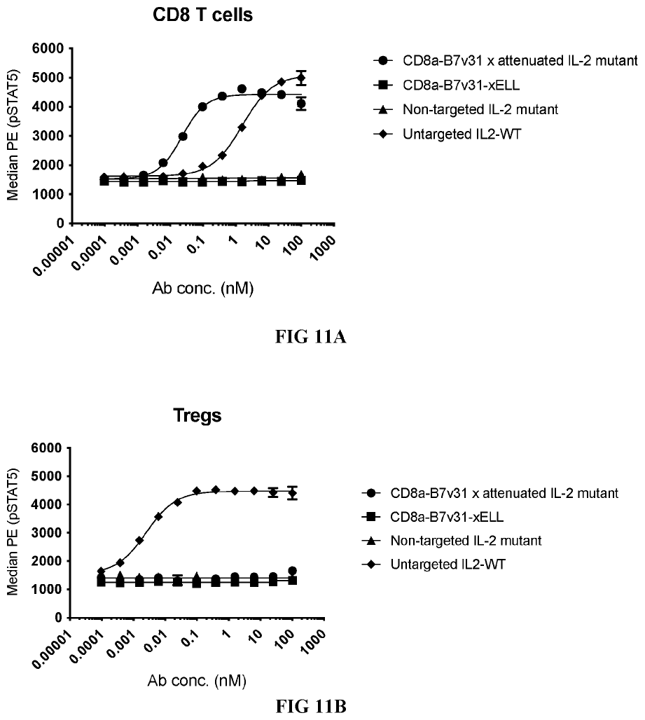

Binding of four polypeptides comprising a humanized CD8a-binding VHH domain fused to an xELL fc region was assessed by flow cytometry on isolated human T cells and on isolated human or cynomolgus monkey peripheral blood mononuclear cells (PBMC). The isolated cells were plated in a 96-well plate at 200,000 cells per well for cynomolgus monkey PBMC, and 100,000 cells per well for human T cells in FACS buffer (PBS, 1% BSA, 0.1% NaN₃, pH 7.4). Test polypeptides were then diluted to 2× the final concentration of 25 nM or 50 nM, and a 3- or 5-fold serial dilution was prepared. FACS buffer alone was used as a secondary antibody-only control. Polypeptide dilutions were added to an equal volume of cells, and assay plates were incubated for 30 minutes at 4° C. After washing twice with 150 μL of FACS buffer per well, the cells were resuspended in FACS buffer with a fluorescently labeled anti-human IgG antibody diluted 1:1000 to detect CD8α binding, fluorescently labeled anti-CD3 antibody (clone SP34.2, 1:50, for PBMC preparations only), and fluorescently labeled anti-CD4 antibody (clone OKT4, 1:100). Propidium iodide (PI) was added at 1:2000 to distinguish live cells from dead cells. Assay plates were incubated at 4° C. for 30 minutes. After one additional wash with 150 μL of FACS buffer CD8α binding was detected by flow cytometry on PI−(CD3+) CD4− cells. Binding was measured on these cell populations as mean fluorescence intensity (MFI) at 647 nm. Flow cytometric detection was performed on an ACEA Biosciences Novocyte-Quanteon Flow Cytometer. The data were plotted and analyzed using GraphPad Prism analysis software. The results are shown in Table 12 and 13 and in FIGS. 10A-D.

TABLE 12
Binding to human CD8 T cells (CD4− enriched T cells)

| Fusion Protein | Bmax (MFI) | K_{d} (nM) | SEQ ID NOs. |
|---|---|---|---|
| hzB7v15-xELL-Fc | 287001 | 0.09133 | 25, 33 |
| hzB7v29-xELL-Fc | 292513 | 0.07587 | 90, 33 |
| hzB7v31-xELL-Fc | 269349 | 0.09089 | 92, 33 |
| hzB7v35-xELL-Fc | 278470 | 0.07665 | 99, 33 |
| hzB7v41-xELL-Fc | 363856 | 0.1093 | 100, 33 |

TABLE 13
Binding on cynomolgus monkey CD8 T cells
(CD3+ CD4− PBMC)

| Fusion Protein | Bmax (MFI) | K_{d} (nM) | SEQ ID NOs. |
|---|---|---|---|
| hzB7v15-xELL-Fc | 121170 | 0.02514 | 25, 33 |
| hzB7v29-xELL-Fc | 123694 | 0.03172 | 90, 33 |
| hzB7v31-xELL-Fc | 103591 | 0.03887 | 92, 33 |
| hzB7v35-xELL-Fc | 110657 | 0.02359 | 99, 33 |
| hzB7v41-xELL-Fc | 99723 | 0.04993 | 100, 33 |

As shown in FIGS. 10A and 10E, as well as Table 12, the tested CD8a-binding polypeptides bound human CD8 T cells with affinities at or below 0.1 nM. FIGS. 10B and 10F show that these polypeptides did not bind to human CD4 T cells. As shown in FIG. 10C and Table 13, the tested CD8a-binding polypeptides bound cynomolgus CD8 T cells with affinities below 0.04 nM. FIGS. 10D and 10H show that the polypeptides did not bind to cynomolgus CD4 T cells.

### Example 9: Specific IL-2 Signaling Induced by a Polypeptide Comprising a CD8a-Binding VHH and an Attenuated IL-2

CD8a-targeted IL-2 activity of a polypeptide comprising a CD8-binding VHH domain (hzB7v31, SEQ ID NO: 92, or hzB7v41, SEQ ID NO: 100), an Fc region, and a mutant, attenuated IL-2 fused to the C-terminus of the Fc region was assessed in a pSTAT5 assay. Control proteins included a polypeptide comprising a CD8a-binding VHH domain and an Fc region but no IL-2, a fusion protein comprising a non-targeting VHH, an Fc region, and a mutant, attenuated IL-2, and wild type IL-2. Increases in levels of phosphorylated STAT5 (pSTAT5) or the percentage of cells expressing pSTAT5 were measured by intracellular flow cytometry as proximal readout of IL-2 receptor engagement and signaling. Enriched human T cells were plated in a 96-well plate at 500,000 cells per well in complete growth media (RPMI, 10% FBS, 1% anti-anti). Test polypeptides were then diluted to 2× the final concentration of 200 nM or 50 nM and a 4-fold serial dilution was made. Serial dilutions were added to the cells and incubated for 15 minutes at 37° C. Cells were then fixed in 100 μL of Cytofix fixation buffer (BD) for 30 minutes at 4° C. Cells were then washed once in 200 μL FACS buffer and permeabilized in Perm buffer III (BD Phosflow) for 30 minutes at 4° C. Permeabilized cells were washed a total of three times in 1× Permeabilization Buffer (eBioscience) and then incubated in 1× Permeabilization Buffer containing fluorescently labeled antibodies against CD4 (OKT4, 1:100), CD3 (SP34-2, 1:50), FoxP3 (236A/E7, 1:40), pSTAT5 (SRBCZX, 1:70), CD25 (M-A251, 1:500) and CD8 (RPA-T8, 1:4000) overnight at 4° C. The next day cells were washed with 150 μL FACS buffer and analyzed using an ACEA Biosciences Novocyte-Quanteon Flow Cytometer. IL-2 signaling was quantified via increases in the median fluorescence intensity or the percentage of positive cells stained with a fluorescently labeled antibody detecting pSTAT5 on CD8 T cells (CD3+CD8+) or regulatory T cells (Tregs, CD3+CD4+FoxP3+). The data were plotted and analyzed using GraphPad Prism analysis software.

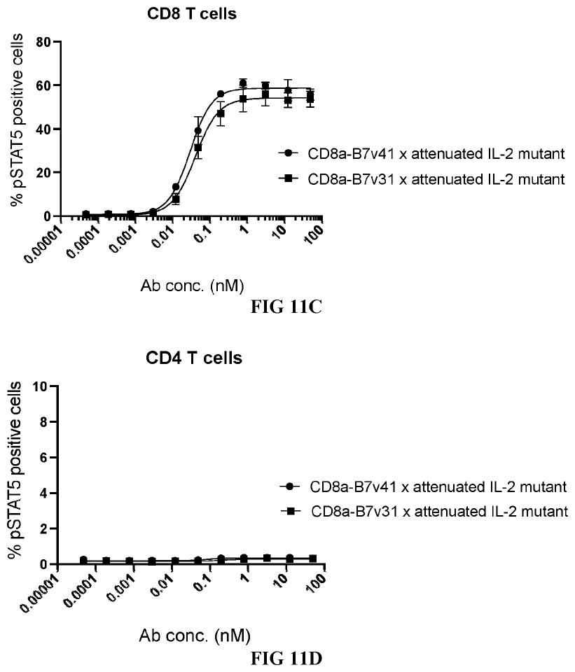

As shown in FIGS. 11A and 11C, the tested polypeptide comprising CD8a-binding VHH hzB7v31 or VHH hzB7v41 domain, an Fc region, and a mutant, attenuated IL-2 fused to the C-terminus of the Fc region induced increasing levels of pSTAT5 or higher percentages of pSTAT5 positive CD8 T cells in a concentration-dependent manner and with an EC₅₀ below 0.03 nM. Wild type IL-2 (untargeted) exhibited about 50-fold less potent activity, with an EC₅₀ of approximately 1.6 nM. Wild type IL2 also induced IL-2 receptor signaling on Tregs with an EC₅₀ of approximately 2.5 pM, whereas no detectable increases in Treg pSTAT5 or percentages of pSTAT5 positive CD4 T cells were induced by the CD8a-targeted attenuated IL-2 (FIGS. 11A-11D). Neither the polypeptide comprising CD8a-hzB7v31 without an IL-2 nor a polypeptide comprising a non-targeted VHH, Fc region, and the mutant, attenuated IL-2 induced detectable increases in pSTAT5 levels in any of the tested cell types, indicating that the attenuated IL-2 required targeting to a cell in order to induce IL-2 receptor signaling activity (FIGS. 11A-11D).

### Example 10: T Cell Proliferation of Human Tumor-Infiltrating T Cells and Healthy Donor T Cells Induced by Polypeptides Comprising a CD8a-Binding VHH and an Attenuated IL-2

The activity of the polypeptide comprising CD8a-binding VHH hzB7v31 domain, an Fc region, and a mutant, attenuated IL-2 fused to the C-terminus of the Fc region was further assessed in a proliferation assay with dissociated tumor cell (DTC) samples from human cancer patients or PBMC from healthy human donor blood. DTC single cell suspensions were generated from biopsies of head and neck, kidney or colon tumors using a human tumor dissociation kit (Miltenyi Biotec). DTC or PBMC were then labeled with the proliferative dye CellTrace Violet (Thermo) according to the manufacturer's recommended protocol. Cells were incubated in complete growth media (RPMI, 10% FBS, 1% anti-anti) supplemented with 10 nM of the test polypeptides or 5-fold dilutions of the test polypeptides starting from a concentration of 200 nM. Control proteins included a polypeptide comprising CD8a-hzB7v31 formatted as VHH-hIgG1-xELL Fc, a fusion protein comprising a non-targeting VHH-hIgG1-xELL Fc and the mutant, attenuated IL-2, and wild type IL-2. After six or seven days in culture, cell subpopulations were labeled with fluorescently tagged antibodies against CD3 (Hit3, 1:100), CD4 (OKT4, 1:200), CD8 (RPA-T8, 1:200), and CD45 (HI30, 1:100), as well as with propidium iodide (PI, 1:2000) to distinguish live cells and dead cells. T cells were classified as CD45+CD3+PI− cells that express either CD4 or CD8a. The cell numbers of these T cell subpopulations were quantified on day six or seven using flow cytometry. Flow cytometric detection was performed on an ACEA Biosciences Novocyte-Quanteon Flow Cytometer. The data were plotted and analyzed using GraphPad Prism analysis software. Cell numbers were normalized to the samples treated with CD8a-hzB7v31-Fc-xELL to determine the fold increase in cell counts over a control polypeptide that does not comprise an IL-2 and does not cause cell proliferation. Percent proliferation was determined by quantifying the percent of cells with lower CellTrace Violet fluorescence intensity than the parental, undivided cell peak.

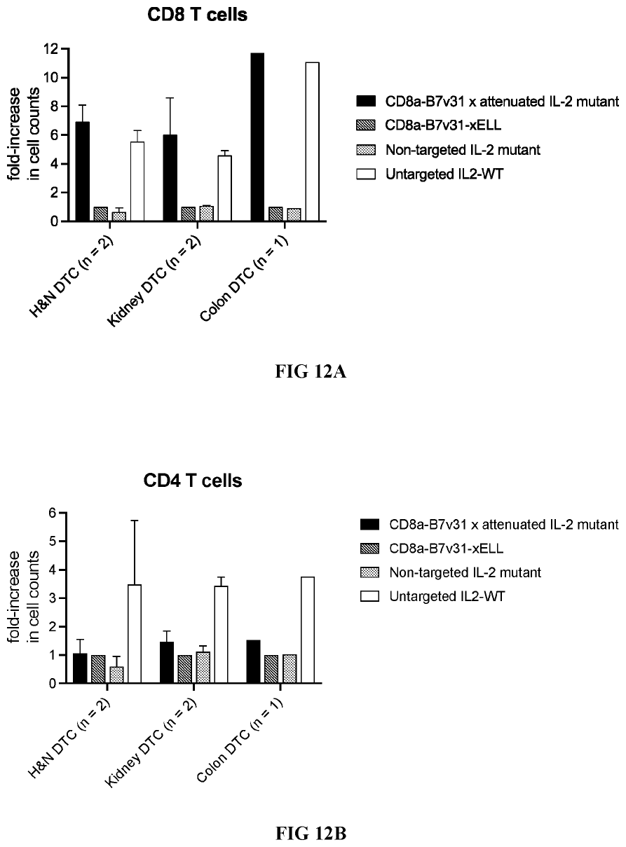

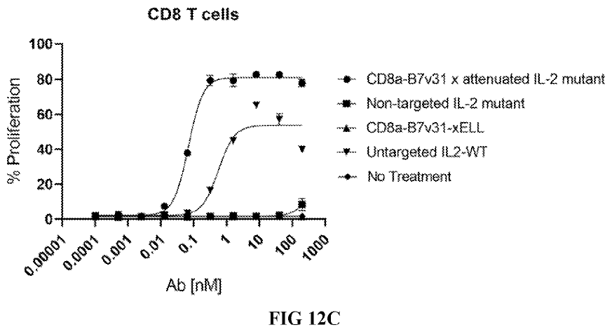

As shown in FIGS. 12A and 12C, the tested polypeptide comprising CD8a-binding VHH hzB7v31 domain, an Fc region, and a mutant, attenuated IL-2 fused to the C-terminus of the Fc region induced proliferation of CD8 T cells in dissociated tumor samples and healthy PBMC. Wild type IL2 also induced proliferation of both CD8 (FIGS. 12A and 12C) and CD4 (FIG. 12B) T cells, whereas the CD8α targeted mutant, attenuated IL-2 did not induce proliferation of CD4 T cells. Neither the polypeptide comprising CD8a-hzB7v31 without an IL-2 nor a polypeptide comprising a non-targeted VHH, Fc region, and the mutant, attenuated IL-2 induced detectable increases in proliferation of CD8 or CD4 T cells, indicating that the attenuated IL-2 required targeting to a cell in order to induce IL-2 receptor signaling activity, such as proliferation.

### Example 11: Cell Expansion of Cynomolgus PBMC Subpopulations Induced by Polypeptides Comprising a CD8a-Binding VHH and an Attenuated IL-2

The effects on in vivo cell expansion of a fusion protein comprising CD8a-binding VHH hzB7v15, an xELL P329G, knob-in-hole heterodimeric Fc region, and an attenuated IL-2 fused to the C-terminus of the “knob” Fc were tested in non-human primates. Cynomolgus monkeys were administered an intravenous bolus injection of the fusion protein at 1.0 mg/kg. Whole blood samples were collected from the study animals before and seven days after fusion protein administration. PBMC from each time point were isolated using density centrifugation in Lymphoprep™ and cells were stained with fluorescently labeled cell type-specific antibody combinations. T cells were classified as CD3+ cells expressing CD4 or CD8α that did not express the B cell marker CD20. Regulatory T cells (“Tregs”) were defined as CD4+ T cells that also expressed CD25 and had reduced levels of CD127. CD4+ conventional T cells (“CD4+ Tcon”) were defined as CD4+ T cells that did not express CD25 and had normal levels of CD127. NK cells were defined as non-T and non-B cells expressing NKG2A and were either positive or negative for CD16. The population staining positive for CD20 was classified as B cells. Absolute cell counts of each PBMC subpopulation were determined using flow cytometry and fold-expansion was calculated by dividing the absolute cell count 7 days post dose by the baseline count pre-dose. Ki67 expression was measured in the PBMC subpopulations described above using additional fixation, permeabilization and staining steps. In brief, cells were stained with fluorescently labeled cell type-specific antibody combinations for the cell surface markers, then fixed and permeabilized using the FoxP3 Transcription Factor Staining Buffer Set (eBioscience). FoxP3 and Ki67 were then detected with specific fluorescently labeled antibodies. T cells were classified as CD3+ cells expressing CD4 or CD8α that did not express the NK cell marker NKG2A. Regulatory T cells (“Tregs”) were defined as CD4+ T cells that also expressed CD25 and FoxP3. CD4+ conventional T cells (“CD4+ Tcon”) were defined as CD4+ T cells that did not express CD25 or FoxP3. NK cells were defined as non-T and expressing NKG2A and were either positive or negative for CD16. Flow cytometric detection was performed on an ACEA Biosciences Novocyte-Quanteon Flow Cytometer. The data were plotted and analyzed using GraphPad Prism analysis software. Fold change was calculated by dividing the cell count per mL of whole blood on day seven by the cell count per mL of whole blood at baseline (pre-dosing).

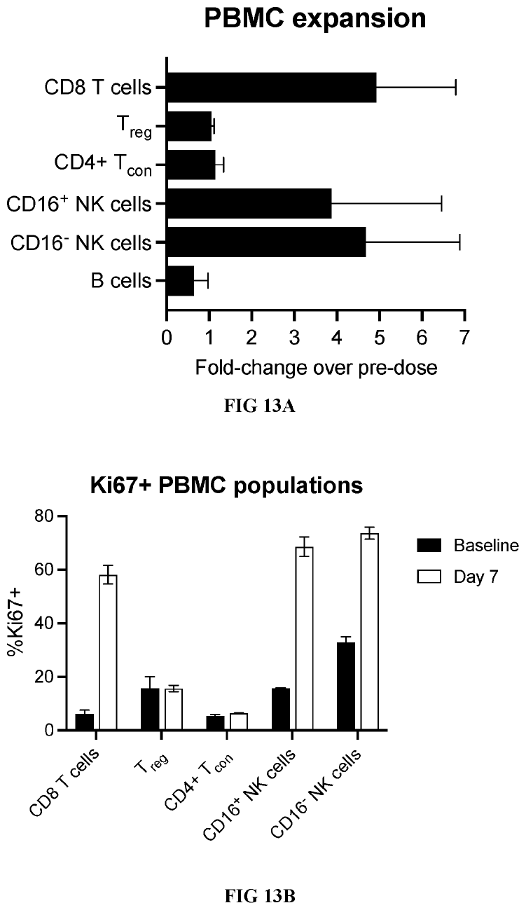

As shown in FIG. 13A, a single dose of the CD8a-targeted attenuated IL-2 at 1 mg/kg resulted in a 5-fold expansion of CD8 T cells, as well as a 3.9-fold and 4.7-fold expansion of CD8a-expressing CD16+ or CD16-NK cells, respectively. Numbers of CD8α negative cell populations, including Tregs, CD4+ conventional T cells and B cells, did not significantly increase between the pre-dose blood draw and day seven. FIG. 13B shows that the specific expansion of CD8a-expressing cell populations in vivo was accompanied by a specific increase in the proliferative marker Ki67. The percentage of Ki67+ proliferating CD8 T cells increased from 6% at baseline to 58% on day seven, while CD16+ and CD16-NK cell populations showed an average increase in Ki67+ cells of 40-53% in the same time frame. The percentage of Ki67+ populations within CD8α negative cell populations including Tregs and CD4+ conventional T cells did not change. These data show that CD8a-targeted attenuated IL-2 specifically induced cell proliferation of CD8α positive cell populations in vivo.

### Example 12: Enhancement of Cytotoxic Activity of CD8 T Cells and Antibody-Dependent Cellular Cytotoxicity Against Human Cancer Cells Induced by Polypeptides Comprising a CD8a-Binding VHH and an Attenuated IL-2

The activity of the fusion comprising CD8a-binding VHH hzB7v31 domain, an xELL knob-in-hole heterodimeric Fc region, and an attenuated IL-2 comprising fused to the C-terminus of the “knob” Fc region was further assessed in tumor cell killing assays with enriched CD8 T cells and in an antibody-dependent cellular cytotoxicity (ADCC) assay in combination with cetuximab. Control proteins included a fusion protein comprising a non-targeting VHH-hIgG1-xELL Fc and the mutant, attenuated IL-2, and wild type IL-2. For the CD8 T cell killing assay, PBMC from healthy human donor blood were used to isolate CD8 T cells and enriched cells were stimulated for 3 days with an antibody against CD3 (clone: OKT3) coated at 1 μg/mL on a culture plate in the presence or absence of additional cytokine support from wild-type IL-2 or the fusion protein comprising the CD8a-binding VHH hzB7v31-hIgG1-xELL Fc and the mutant, attenuated IL-2 (each at 1 nM). On the day of the target cell killing assay, A431 cells were labeled with CYTO-ID red long-term cell tracer (Enzo) then plated at 4,000 cells per well in 100 μL in a 96-well flat-bottom plate and allowed to adhere for 4 hours. Pre-stimulated CD8 T cells were washed once in PBS and added to the labeled A431 target cells at different effector-to-target cell ratios (20:1, 10:1 and 5:1) as indicated. Caspase-3/7 Green Dye (Sartorius) was added to each well to detect cell death. A431 killing was determined after 20 h by quantifying the overlap of Caspase-3/7 and CYTO-ID red using an Incucyte imager.

For the ADCC assay, A431 cells were labeled with CYTO-ID red long-term cell tracer (Enzo) then plated at 10,000 cells per well in 100 μL in a 96-well flat-bottom plate and allowed to adhere for 4 hours. Human PBMC were thawed and tested for NK cell frequency by flow cytometry. To each well, 25 μL of Incucyte® Caspase-3/7 Green Dye for Apoptosis (Sartorius) for a final dilution of 1:2000, 25 L of media or the ADCC antibody cetuximab at a final concentration of 20 nM, 25 μL of media, wild type recombinant IL-2 at a final concentration of 1 nM, or IL-2 variant fusion polypeptides at a final concentration of 1 nM, and 25 μL of human PBMC adjusted to a concentration of 10 or 5 NK cells per 1 A431 cell. Cells were allowed to settle at room temperature for 10 minutes, then the plate was placed in an Incucyte imager at 37° C. for imaging. A431 killing was determined after 15 h by quantifying the overlap of Caspase-3/7 and CYTO-ID red, with maximal killing defined by 20 nM cetuximab. All data were plotted and analyzed using GraphPad Prism analysis software.

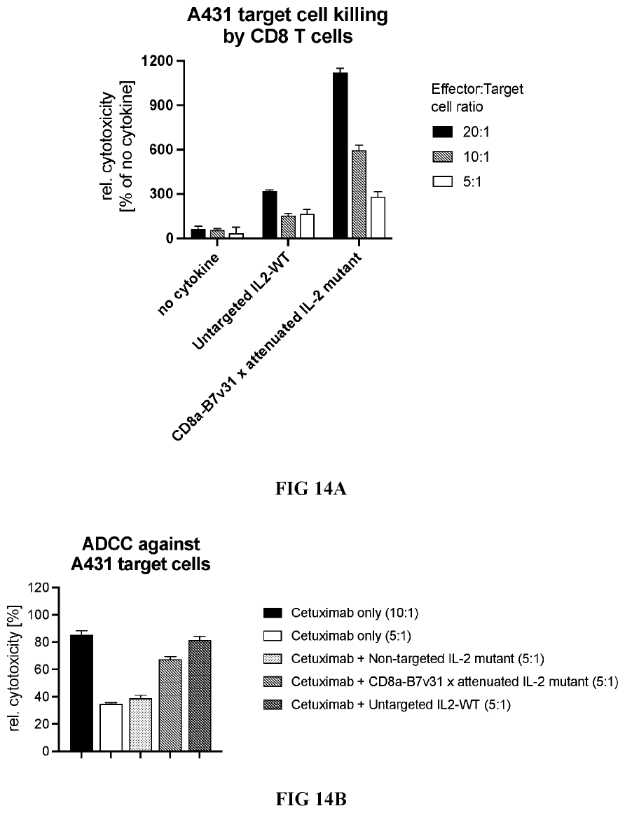

As shown in FIGS. 14A and 14B, the tested polypeptide comprising CD8a-binding VHH hzB7v31 domain, an Fc region, and a mutant, attenuated IL-2 fused to the C-terminus of the Fc region enhanced the relative cytotoxicity of CD8 T cells at different effector-to-target cell ratios (FIG. 14A) and helped improve the Cetuximab-driven ADCC activity of PBMC against EGFR positive A431 target cells at suboptimal effector-to-target cell ratios (FIG. 14B). The extent of the activity with CD8 T cells was 3 to 4-fold higher than that observed with wild type IL-2, but comparable in the ADCC assay. A fusion protein comprising a non-targeting VHH-hIgG1-xELL Fc and the mutant, attenuated IL-2 was not able to improve the ADCC activity of a lower effector-to-target cell ratio, indicating that the attenuated IL-2 required targeting to an effector cell in order to induce IL-2 receptor signaling activity and enhanced cytotoxicity.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE of
Certain Sequences

| SEQ | | |
|---|---|---|
| ID NO | Description | Sequence |
| 1 | Human CD8a, precursor | MALPVTALLL PLALLLHAAR PSQFRVSPLD RTWNLGETVE |
| | (signal sequence is amino | LKCQVLLSNP TSGCSWLFQP RGAAASPTFL LYLSQNKPKA |
| | acids 1-21) | AEGLDTQRFS GKRLGDTFVL TLSDFRRENE GYYFCSALSN |
| | | SIMYFSHFVP VFLPAKPTTT PAPRPPTPAP TIASQPLSLR |
| | | PEACRPAAGG AVHTRGLDFA CDIYIWAPLA GTCGVLLLSL |
| | | VITLYCNHRN RRRVCKCPRP VVKSGDKPSL SARYV |
| 72 | CD8a-IgV LFc antigen for | MALPVTALLL PLALLLHAAR PSQFRVSPLD RTWNLGETVE |
| | immunization (signal | LKCQVLLSNP TSGCSWLFQP RGAAASPTFL LYLSQNKPKA |
| | sequence is amino acids 1- | AEGLDTQRFS GKRLGDTFVL TLSDFRRENE GYYFCSALSN |
| | 21) | SIMYFSHFVP VFLPAKTGGS GGGGCPPCPA PELPGGPSVF |
| | | VFPPKPKDVL SISGRPEVTC VVVDVGKEDP EVNFNWYIDG |
| | | VEVRTANTKP KEEQFNSTYR VVSVLPIQHQ DWLTGKEFKC |
| | | KVNNKALPAP IERTISKAKG QTREPQVYTL APHREELAKD |
| | | TVSVTCLVKG FYPADINVEW QRNGQPESEG TYANTPPQLD |
| | | NDGTYFLYSK LSVGKNTWQR GETLTCVVMH EALHNHYTQK |
| | | SISQSLGK |
| 2 | B7 VHH | QVQLVQSGGGLVRPGGSLRLSCAASGFTFDDYAMSWVRQAPGK |
| | | GLEWVSTITWDGEGTDYAESMKGRFTISRDNAKSTVYLQMNGL |
| | | KSEDTAVYVCAKGSPELQYDSWGQGTQVTVKPXX |
| | | wherein each X is Gly or is absent |
| 3 | CDR1 of B7, hzB7v1, | GFTFDDYAMS |
| | hzB7v2, hzB7v3, hzB7v4, | |
| | hzB7v5, hzB7v6, hzB7v7, | |
| | hzB7v8, hzB7v9, hzB7v10, | |
| | hzB7v11, hzB7v12, | |
| | hzB7v13, hzB7v14, | |
| | hzB7v15, hzB7v16, | |
| | hzB7v17 hzB7v18, | |
| | hzB7v19, hzB7v20, | |
| | hzB7v21, hzB7v25, | |
| | hzB7v26, hzB7v27, | |
| | hzB7v28, hzB7v29, | |
| | hzB7v30, hzB7v31, | |
| | hzB7v33, hzB7v34, | |
| | hzB7v35, hzB7v36, | |
| | hzB7v37, hzB7v39, and | |
| | hzB7v40 | |
| 73 | CDR1 of hzB7v23 | GFTFDSYAMS |
| 74 | CDR1 of hzB7v24 | GFTFSSYAMS |
| 4 | CDR2 of B7, hzB7v1, | TITWDGEGTD |
| | hzB7v2, hzB7v3, hzB7v4, | |
| | hzB7v5, hzB7v8, hzB7v9, | |
| | hzB7v13, and hzB7v14 | |
| 12 | CDR2 of hzB7v6 and | TITWEGEGTD |
| | hzB7v10 | |
| 14 | CDR2 of hzB7v7, hzB7v11, | TITWDAEGTD |
| | hzB7v15, hzB7v19, | |
| | hzB7v20, hzB7v21, | |
| | hzB7v23, hzB7v24, and | |
| | hzB7v28 | |
| 22 | CDR2 of hzB7v12 | TITWSGEGTD |
| 27 | CDR2 of hzB7v16 | TITWEGESTD |
| 29 | CDR2 of hzB7v17 | TITWEGEGTY |
| 31 | CDR2 of hzB7v18 | TITWEGESTY |
| 75 | CDR2 of hzB7v25 | TITWSAEGTD |
| 76 | CDR2 of hzB7v26, | TITWDAGGTD |
| | hzB7v30, and hzB7v36 | |
| 77 | CDR2 of hzB7v27, | TITWDAEGTY |
| | hzB7v29, and hzB7v35 | |
| 78 | CDR2 of hzB7v31 and | TITWDAGGTY |
| | hzB7v37 | |
| 79 | CDR2 of hzB7v33 and | TITWSAEGTY |
| | hzB7v39 | |
| 80 | CDR2 of hzB7v34 and | TITWSAGGTD |
| | hzB7v40 | |
| 5 | CDR3 of B7, hzB7v1, | GSPELQYDS |
| | hzB7v2, hzB7v3, hzB7v4, | |
| | hzB7v5, hzB7v6, hzB7v7, | |
| | hzB7v10, hzB7v11, | |
| | hzB7v12, hzB7v16, | |
| | hzB7v17, and hzB7v18 | |
| 16 | CDR3 of hzB7v8 and | GSPELQYES |
| | hzB7v13 | |
| 18 | CDR3 of hzB7v9, hzB7v14, | GSPELQYDT |
| | hzB7v15, hzB7v19, | |
| | hzB7v20, hzB7v21, | |
| | hzB7v23, hzB7v24, | |
| | hzB7v25, hzB7v26, | |
| | hzB7v27, hzB7v28, | |
| | hzB7v29, hzB7v30, | |
| | hzB7v31, hzB7v33, | |
| | hzB7v34, hzB7v35, | |
| | hzB7v36, hzB7v37, | |
| | hzB7v39, and hzB7v40 | |
| 6 | hzB7v1 VHH | EVQLVESGGGEVQPGGSLRLSCAASGFTFDDYAMSWVRQAPGK |
| | | GLEWVSTITWDGEGTDYAESVKGRFTISRDNAKNTLYLQMSSL |
| | | RAEDTAVYYCAKGSPELQYDSWGQGTLVTVKPXX |
| | | wherein each X is Gly or is absent |
| 7 | hzB7v2 VHH | EVQLVESGGGEVQPGGSLRLSCAASGFTFDDYAMSWVRQAPGK |
| | | GLEWVSTITWDGEGTDYAESVKGRFTISRDNAKNTVYLQMSSL |
| | | RAEDTAVYYCAKGS PELQYDSWGQGTLVTVKPXX |
| | | wherein each X is Gly or is absent |
| 8 | hzB7v3 VHH | EVQLVESGGGEVQPGGSLRLSCAASGFTFDDYAMSWVRQAPGK |
| | | GLEWVSTITWDGEGTDYAESMKGRFTISRDNAKNTVYLQMSSL |
| | | RAEDTAVYYCAKGSPELQYDSWGQGTLVTVKPXX |
| | | wherein each X is Gly or is absent |
| 9 | hzB7v4 VHH | EVQLVESGGGEVQPGGSLRLSCAASGFTFDDYAMSWVRQAPGK |
| | | GLEWVSTITWDGEGTDYAESMKGRFTISRDNAKSTVYLQMSSL |
| | | RAEDTAVYYCAKGSPELQYDSWGQGTLVTVKPXX |
| | | wherein each X is Gly or is absent |
| 10 | hzB7v5 VHH | EVQLVESGGGEVQPGGSLRLSCAASGFTFDDYAMSWVRQAPGK |
| | | GLEWVSTITWDGEGTDYAESMKGRFTISRDNAKSTVYLQMSSL |
| | | RAEDTAVYVCAKGSPELQYDSWGQGTLVTVKPXX |
| | | wherein each X is Gly or is absent |
| 11 | hzB7v6 VHH | EVQLVESGGGEVQPGGSLRLSCAASGFTFDDYAMSWVRQAPGK |
| | | GLEWVSTITWEGEGTDYAESMKGRFTISRDNAKSTVYLQMSSL |
| | | RAEDTAVYVCAKGSPELQYDSWGQGTLVTVKPXX |
| | | wherein each X is Gly or is absent |
| 13 | hzB7v7 VHH | EVQLVESGGGEVQPGGSLRLSCAASGFTFDDYAMSWVRQAPGK |
| | | GLEWVSTITWDAEGTDYAESMKGRFTISRDNAKSTVYLQMSSL |
| | | RAEDTAVYVCAKGSPELQYDSWGQGTLVTVKPXX |
| | | wherein each X is Gly or is absent |
| 15 | hzB7v8 VHH | EVQLVESGGGEVQPGGSLRLSCAASGFTFDDYAMSWVRQAPGK |
| | | GLEWVSTITWDGEGTDYAESMKGRFTISRDNAKSTVYLQMSSL |
| | | RAEDTAVYVCAKGSPELQYESWGQGTLVTVKPXX |
| | | wherein each X is Gly or is absent |
| 17 | hzB7v9 VHH | EVQLVESGGGEVQPGGSLRLSCAASGFTFDDYAMSWVRQAPGK |
| | | GLEWVSTITWDGEGTDYAESMKGRFTISRDNAKSTVYLQMSSL |
| | | RAEDTAVYVCAKGSPELQYDTWGQGTLVTVKPXX |
| | | wherein each X is Gly or is absent |
| 19 | hzB7v10 VHH | EVQLVESGGGEVQPGGSLRLSCAASGFTFDDYAMSWVRQAPGK |
| | | GLEWVSTITWEGEGTDYAESVKGRFTISRDNAKNTVYLQMSSL |
| | | RAEDTAVYYCAKGSPELQYDSWGQGTLVTVKPGG |
| 20 | hzB7v11 VHH | EVQLVESGGGEVQPGGSLRLSCAASGFTFDDYAMSWVRQAPGK |
| | | GLEWVSTITWDAEGTDYAESVKGRFTISRDNAKNTVYLQMSSL |
| | | RAEDTAVYYCAKGSPELQYDSWGQGTLVTVKPXX |
| | | wherein each X is Gly or is absent |
| 21 | hzB7v12 VHH | EVQLVESGGGEVQPGGSLRLSCAASGFTFDDYAMSWVRQAPGK |
| | | GLEWVSTITWSGEGTDYAESVKGRFTISRDNAKNTVYLQMSSL |
| | | RAEDTAVYYCAKGSPELQYDSWGQGTLVTVKPXX |
| | | wherein each X is Gly or is absent |
| 23 | hzB7v13 VHH | EVQLVESGGGEVQPGGSLRLSCAASGFTFDDYAMSWVRQAPGK |
| | | GLEWVSTITWDGEGTDYAESVKGRFTISRDNAKNTVYLQMSSL |
| | | RAEDTAVYYCAKGSPELQYESWGQGTLVTVKPXX |
| | | wherein each X is Gly or is absent |
| 24 | hzB7v14 VHH | EVQLVESGGGEVQPGGSLRLSCAASGFTFDDYAMSWVRQAPGK |
| | | GLEWVSTITWDGEGTDYAESVKGRFTISRDNAKNTVYLQMSSL |
| | | RAEDTAVYYCAKGSPELQYDTWGQGTLVTVKPXX |
| | | wherein each X is Gly or is absent |
| 25 | hzB7v15 VHH | EVQLVESGGGEVQPGGSLRLSCAASGFTFDDYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKPXX |
| | | wherein each X is Gly or is absent |
| 26 | hzB7v16 VHH | EVQLVESGGGEVQPGGSLRLSCAASGFTFDDYAMSWVRQAPGK |
| | | GLEWVSTITWEGESTDYAESVKGRFTISRDNAKNTVYLQMSSL |
| | | RAEDTAVYYCAKGSPELQYDSWGQGTLVTVKPXX |
| | | wherein each X is Gly or is absent |
| 28 | hzB7v17 VHH | EVQLVESGGGEVQPGGSLRLSCAASGFTFDDYAMSWVRQAPGK |
| | | GLEWVSTITWEGEGTYYAESVKGRFTISRDNAKNTVYLQMSSL |
| | | RAEDTAVYYCAKGSPELQYDSWGQGTLVTVKPGG |
| 30 | hzB7v18 VHH | EVQLVESGGGEVQPGGSLRLSCAASGFTFDDYAMSWVRQAPGK |
| | | GLEWVSTITWEGESTYYAESVKGRFTISRDNAKNTVYLQMSSL |
| | | RAEDTAVYYCAKGSPELQYDSWGQGTLVTVKPXX |
| | | wherein each X is Gly or is absent |
| 81 | hzB7v19 VHH | QVQLVESGGGEVQPGGSLRLSCAASGFTFDDYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKP |
| 82 | hzB7v20 VHH | EVQLVQSGGGEVQPGGSLRLSCAASGFTFDDYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKP |
| 83 | hzB7v21 VHH | QVQLVQSGGGEVQPGGSLRLSCAASGFTFDDYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKP |
| 84 | hzB7v23 VHH | QVQLVQSGGGEVQPGGSLRLSCAASGFTFDSYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKP |
| 85 | hzB7v24 VHH | QVQLVQSGGGEVQPGGSLRLSCAASGFTFSSYAMSWVRQAPGK |
| | | GLEWVSTITWDAEGTDYAESVKGRFTISRDNAKNTVYLQMSSL |
| | | RAEDTAVYYCAKGSPELQYDTWGQGTLVTVKP |
| 86 | hzB7v25 VHH | QVQLVQSGGGEVQPGGSLRLSCAASGFTFDDYAMSWVRQAPGK |
| | | GLEWVSTITWSAEGTDYAESVKGRFTISRDNAKNTVYLQMSSL |
| | | RAEDTAVYYCAKGSPELQYDTWGQGTLVTVKP |
| 87 | hzB7v26 VHH | QVQLVQSGGGEVQPGGSLRLSCAASGFTFDDYAMSWVRQAPGK |
| | | GLEWVSTITWDAGGTDYAESVKGRFTISRDNAKNTVYLQMSSL |
| | | RAEDTAVYYCAKGSPELQYDTWGQGTLVTVKP |
| 88 | hzB7v27 VHH | QVQLVQSGGGEVQPGGSLRLSCAASGFTFDDYAMSWVRQAPGK |
| | | GLEWVSTITWDAEGTYYAESVKGRFTISRDNAKNTVYLQMSSL |
| | | RAEDTAVYYCAKGSPELQYDTWGQGTLVTVKP |
| 89 | hzB7v28 VHH | QVQLVQSGGGEVQPGGSLRLSCAASGFTFDDYAMSWVRQAPGK |
| | | GLEWVSTITWDAEGTDYAAPVKGRFTISRDNAKNTVYLQMSSL |
| | | RAEDTAVYYCAKGSPELQYDTWGQGTLVTVKP |
| 90 | hzB7v29 VHH | QVQLVQSGGGEVQPGGSLRLSCAASGFTFDDYAMSWVRQAPGK |
| | | GLEWVSTITWDAEGTYYAAPVKGRFTISRDNAKNTVYLQMSSL |
| | | RAEDTAVYYCAKGSPELQYDTWGQGTLVTVKP |
| 91 | hzB7v30 VHH | QVQLVQSGGGEVQPGGSLRLSCAASGFTFDDYAMSWVRQAPGK |
| | | GLEWVSTITWDAGGTDYAAPVKGRFTISRDNAKNTVYLQMSSL |
| | | RAEDTAVYYCAKGSPELQYDTWGQGTLVTVKP |
| 92 | hzB7v31 VHH | QVQLVQSGGGEVQPGGSLRLSCAASGFTFDDYAMSWVRQAPGK |
| | | GLEWVSTITWDAGGTYYAAPVKGRFTISRDNAKNTVYLQMSSL |
| | | RAEDTAVYYCAKGSPELQYDTWGQGTLVTVKP |
| 93 | hzB7v33 VHH | QVQLVQSGGGEVQPGGSLRLSCAASGFTFDDYAMSWVRQAPGK |
| | | GLEWVSTITWSAEGTYYAAPVKGRFTISRDNAKNTVYLQMSSL |
| | | RAEDTAVYYCAKGSPELQYDTWGQGTLVTVKP |
| 94 | hzB7v34 VHH | QVQLVQSGGGEVQPGGSLRLSCAASGFTFDDYAMSWVRQAPGK |
| | | GLEWVSTITWSAGGTDYAAPVKGRFTISRDNAKNTVYLQMSSL |
| | | RAEDTAVYYCAKGSPELQYDTWGQGTLVTVKP |
| 99 | hzB7v35 VHH | QVQLVQSGGGEVKPGGSLRLSCAASGFTFDDYAMSWVRQAPGK |
| | | GLEWVSTITWDAEGTYYAAPVKGRFTISRDNAKNTVYLQMSSL |
| | | RAEDTAVYYCAKGSPELQYDTWGQGTLVTVKP |
| 95 | hzB7v36 VHH | QVQLVQSGGGEVKPGGSLRLSCAASGFTFDDYAMSWVRQAPGK |
| | | GLEWVSTITWDAGGTDYAAPVKGRFTISRDNAKNTVYLQMSSL |
| | | RAEDTAVYYCAKGSPELQYDTWGQGTLVTVKP |
| 96 | hzB7v37 VHH | QVQLVQSGGGEVKPGGSLRLSCAASGFTFDDYAMSWVRQAPGK |
| | | GLEWVSTITWDAGGTYYAAPVKGRFTISRDNAKNTVYLQMSSL |
| | | RAEDTAVYYCAKGSPELQYDTWGQGTLVTVKP |
| 97 | hzB7v39 VHH | QVQLVQSGGGEVKPGGSLRLSCAASGFTFDDYAMSWVRQAPGK |
| | | GLEWVSTITWSAEGTYYAAPVKGRFTISRDNAKNTVYLQMSSL |
| | | RAEDTAVYYCAKGSPELQYDTWGQGTLVTVKP |
| 98 | hzB7v40 VHH | QVQLVQSGGGEVKPGGSLRLSCAASGFTFDDYAMSWVRQAPGK |
| | | GLEWVSTITWSAGGTDYAAPVKGRFTISRDNAKNTVYLQMSSL |
| | | RAEDTAVYYCAKGSPELQYDTWGQGTLVTVKP |
| 100 | hzB7v41 VHH | QVQLVQSGGGEVQPGGSLRLSCAASGFTFDDYAMSWVRQAPGK |
| | | GLEWVSTITWDAGGTYYAAPVKGRFTISRDNAKNTVYLQMSSL |
| | | RAEDTAVYYCAKGSPELQYDTWGQGTLVTVRP |
| 32 | human IgG1 Fc region | DKTHTCPPC PAPELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 33 | human IgG1 Fc xELL | DKTHTCPPCPAPGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPGK |
| 34 | Fc region M252Y and | DKTHTCPPCP APELLGGPSV FLFPPKPKDT LYISRTPEVT |
| | M428V (YV) S354C | CVVVDVSHED PEVKFNWYVD GVEVHNAKTK PREEQYNSTY |
| | T366W knob | RVVSVLTVLH QDWLNGKEYK CKVSNKALPA PIEKTISKAK |
| | | GQPREPQVYT LPPCRDELTK NQVSLWCLVK GFYPSDIAVE |
| | | WESNGQPENN YKTTPPVLDS DGSFFLYSKL TVDKSRWQQG |
| | | NVFSCSVVHE ALHNHYTQKS |
| 35 | Fc region M252Y, M428V, | DKTHTCPPCP APELLGGPSV FLFPPKPKDT LYISRTPEVT |
| | H435R (YVR) T366S, | CVVVDVSHED PEVKFNWYVD GVEVHNAKTK PREEQYNSTY |
| | L368A, Y407V hole | RVVSVLTVLH QDWLNGKEYK CKVSNKALPA PIEKTISKAK |
| | | GQPREPQVCT LPPSRDELTK NQVSLSCAVK GFYPSDIAVE |
| | | WESNGQPENN YKTTPPVLDS DGSFFLVSKL TVDKSRWQQG |
| | | NVFSCSVVHE ALHNRYTQKS LSLSPGK |
| 36 | Fc region xELL H435R | DKTHTC PPCPAPGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 37 | Fc region xELL M252Y and | DKTHTC PPCPAPGGPS VFLFPPKPKD TLYISRTPEV |
| | M428V (YV) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNHYTQK SLSLSPGK |
| 38 | Fc region xELL M252Y and | DKTHTC PPCPAPGGPS VFLFPPKPKD TLYISRTPEV |
| | M428L (YL) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNHYTQK SLSLSPGK |
| 39 | Fc region xELL M252Y, | DKTHTC PPCPAPGGPS VFLFPPKPKD TLYISRTPEV |
| | M428L, H435R (YLR) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNRYTQK SLSLSPGK |
| 40 | Fc region xELL M252Y, | DKTHTC PPCPAPGGPS VFLFPPKPKD TLYISRTPEV |
| | M428V, H435R (YVR) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNRYTQK SLSLSPGK |
| 41 | Fc region xELL S354C | DKTHTC PPCPAPGGPS VFLFPPKPKD TLMISRTPEV |
| | T366W knob | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 42 | Fc region xELL H435R | DKTHTC PPCPAPGGPS VFLFPPKPKD TLMISRTPEV |
| | S354C T366W knob | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 43 | Fc region xELL M252Y and | DKTHTCPPCPAPGGPSVFLFPPKPKDTLYISRTPEVTCVVVDV |
| | M428V (YV) S354C | SHEDPEVKFNWYVDGVEVHNAKTKPREEQYNSTYRVVSVLTVL |
| | T366W knob | HQDWLNGKEYKCKVSNKALPAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVVHEALHNHYTQ |
| | | KSLSLSPGK |
| 44 | Fc region xELL M252Y and | DKTHTCPPCPAPGGPSVFLFPPKPKDTLYISRTPEVTCVVVDV |
| | M428L (YL) S354C | SHEDPEVKFNWYVDGVEVHNAKTKPREEQYNSTYRVVSVLTVL |
| | T366W knob | HQDWLNGKEYKCKVSNKALPAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVLHEALHNHYTQ |
| | | KSLSLSPGK |
| 45 | Fc region xELL M252Y, | DKTHTCPPCPAPGGPSVFLFPPKPKDTLYISRTPEVTCVVVDV |
| | M428L, H435R (YLR) | SHEDPEVKFNWYVDGVEVHNAKTKPREEQYNSTYRVVSVLTVL |
| | S354C T366W knob | HQDWLNGKEYKCKVSNKALPAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVLHEALHNRYTQ |
| | | KSLSLSPGK |
| 46 | Fc region xELL M252Y, | DKTHTCPPCPAPGGPSVFLFPPKPKDTLYISRTPEVTCVVVDV |
| | M428V, H435R (YVR) | SHEDPEVKFNWYVDGVEVHNAKTKPREEQYNSTYRVVSVLTVL |
| | S354C T366W knob | HQDWLNGKEYKCKVSNKALPAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVVHEALHNRYTQ |
| | | KSLSLSPGK |
| 47 | Fc region xELL T366S, | DKTHTCPPCPAPGGPSVFLFPPKPKDTLMISRTPEVTCVVVDV |
| | L368A, Y407V hole | SHEDPEVKFNWYVDGVEVHNAKTKPREEQYNSTYRVVSVLTVL |
| | | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYTQ |
| | | KSLSLSPGK |
| 48 | Fc region xELL H435R, | DKTHTCPPCPAPGGPSVFLFPPKPKDTLMISRTPEVTCVVVDV |
| | T366S, L368A, Y407V hole | SHEDPEVKFNWYVDGVEVHNAKTKPREEQYNSTYRVVSVLTVL |
| | | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNRYTQ |
| | | KSLSLSPGK |
| 49 | Fc region xELL M252Y and | DKTHTCPPCPAPGGPSVFLFPPKPKDTLYISRTPEVTCVVVDV |
| | M428V (YV) T366S, | SHEDPEVKFNWYVDGVEVHNAKTKPREEQYNSTYRVVSVLTVL |
| | L368A, Y407V hole | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVVHEALHNHYTQ |
| | | KSLSLSPGK |
| 50 | Fc region xELL M252Y and | DKTHTCPPCPAPGGPSVFLFPPKPKDTLYISRTPEVTCVVVDV |
| | M428L (YL) T366S, | SHEDPEVKFNWYVDGVEVHNAKTKPREEQYNSTYRVVSVLTVL |
| | L368A, Y407V hole | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVLHEALHNHYTQ |
| | | KSLSLSPGK |
| 51 | Fc region xELL M252Y, | DKTHTCPPCPAPGGPSVFLFPPKPKDTLYISRTPEVTCVVVDV |
| | M428L, H435R (YLR) | SHEDPEVKFNWYVDGVEVHNAKTKPREEQYNSTYRVVSVLTVL |
| | T366S, L368A, Y407V hole | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVLHEALHNRYTQ |
| | | KSLSLSPGK |
| 52 | Fc region xELL M252Y, | DKTHTCPPCPAPGGPSVFLFPPKPKDTLYISRTPEVTCVVVDV |
| | M428V, H435R (YVR) | SHEDPEVKFNWYVDGVEVHNAKTKPREEQYNSTYRVVSVLTVL |
| | T366S, L368A, Y407V hole | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVVHEALHNRYTQ |
| | | KSLSLSPGK |
| 53 | Fc region H435R | DKTHTCPPCP APELLGGPS VFLFPPKPKD TLMISRTPEV |
| | | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 54 | Fc region M252Y and | DKTHTCPPCP APELLGGPS VFLFPPKPKD TLYISRTPEV |
| | M428V (YV) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNHYTQK SLSLSPGK |
| 55 | Fc region M252Y and | DKTHTCPPCP APELLGGPS VFLFPPKPKD TLYISRTPEV |
| | M428L (YL) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNHYTQK SLSLSPGK |
| 56 | Fc region M252Y, M428L, | DKTHTCPPCP APELLGGPS VFLFPPKPKD TLYISRTPEV |
| | H435R (YLR) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVLH EALHNRYTQK SLSLSPGK |
| 57 | Fc region M252Y, M428V, | DKTHTCPPCP APELLGGPS VFLFPPKPKD TLYISRTPEV |
| | H435R (YVR) | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPSRDELT KNQVSLTCLV KGFYPSDIAV |
| | | EWESNGQPEN NYKTTPPVLD SDGSFFLYSK LTVDKSRWQQ |
| | | GNVFSCSVVH EALHNRYTQK SLSLSPGK |
| 58 | Fc region S354C T366W | DKTHTCPPCP APELLGGPS VFLFPPKPKD TLMISRTPEV |
| | knob | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNHYTQK SLSLSPGK |
| 59 | Fc region H435R S354C | DKTHTCPPCP APELLGGPS VFLFPPKPKD TLMISRTPEV |
| | T366W knob | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNRYTQK SLSLSPGK |
| 60 | Fc region M252Y and | DKTHTCPPCPAPELLGGPSVFLFPPKPKDTLYISRTPEVTCVV |
| | M428L (YL) S354C | VDVSHEDPEVKFNWYVDGVEVHNAKTKPREEQYNSTYRVVSVL |
| | T366W knob | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVLHEALHNH |
| | | YTQKSLSLSPGK |
| 61 | Fc region M252Y, M428L, | DKTHTCPPCPAPELLGGPSVFLFPPKPKDTLYISRTPEVTCVV |
| | H435R (YLR) S354C | VDVSHEDPEVKFNWYVDGVEVHNAKTKPREEQYNSTYRVVSVL |
| | T366W knob | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVLHEALHNR |
| | | YTQKSLSLSPGK |
| 62 | Fc region M252Y, M428V, | DKTHTCPPCPAPELLGGPSVFLFPPKPKDTLYISRTPEVTCVV |
| | H435R (YVR) S354C | VDVSHEDPEVKFNWYVDGVEVHNAKTKPREEQYNSTYRVVSVL |
| | T366W knob | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVVHEALHNR |
| | | YTQKSLSLSPGK |
| 63 | Fc region T366S, L368A, | DKTHTCPPCPAPELLGGPSVFLFPPKPKDTLMISRTPEVTCVV |
| | Y407V hole | VDVSHEDPEVKFNWYVDGVEVHNAKTKPREEQYNSTYRVVSVL |
| | | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVMHEALHNH |
| | | YTQKSLSLSPGK |
| 64 | Fc region H435R, T366S, | DKTHTCPPCPAPELLGGPSVFLFPPKPKDTLMISRTPEVTCVV |
| | L368A, Y407V hole | VDVSHEDPEVKFNWYVDGVEVHNAKTKPREEQYNSTYRVVSVL |
| | | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVMHEALHNR |
| | | YTQKSLSLSPGK |
| 65 | Fc region M252Y and | DKTHTCPPCPAPELLGGPSVFLFPPKPKDTLYISRTPEVTCVV |
| | M428V (YV) T366S, | VDVSHEDPEVKFNWYVDGVEVHNAKTKPREEQYNSTYRVVSVL |
| | L368A, Y407V hole | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVVHEALHNH |
| | | YTQKSLSLSPGK |
| 66 | Fc region M252Y and | DKTHTCPPCPAPELLGGPSVFLFPPKPKDTLYISRTPEVTCVV |
| | M428L (YL) T366S, | VDVSHEDPEVKFNWYVDGVEVHNAKTKPREEQYNSTYRVVSVL |
| | L368A, Y407V hole | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVLHEALHNH |
| | | YTQKSLSLSPGK |
| 67 | Fc region M252Y, M428L, | DKTHTCPPCPAPELLGGPSVFLFPPKPKDTLYISRTPEVTCVV |
| | H435R (YLR) T366S, | VDVSHEDPEVKFNWYVDGVEVHNAKTKPREEQYNSTYRVVSVL |
| | L368A, Y407V hole | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVLHEALHNR |
| | | YTQKSLSLSPGK |
| 68 | Fc region xELL P329G, | DKTHTCPPCPAPGGPSVFLFPPKPKDTLMISRTPEVTCVVVDV |
| | H435R, T366S, L368A, | SHEDPEVKFNWYVDGVEVHNAKTKPREEQYNSTYRVVSVLTVL |
| | Y407V hole AK447 | HQDWLNGKEYKCKVSNKALGAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNRYTQ |
| | | KSLSLSPG |
| 69 | Fc region xELL P329G, | DKTHTCPPCPAPGGPSVFLFPPKPKDTLMISRTPEVTCVVVDV |
| | S354C, T366W knob | SHEDPEVKFNWYVDGVEVHNAKTKPREEQYNSTYRVVSVLTVL |
| | AK447 | HQDWLNGKEYKCKVSNKALGAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVMHEALHNHYTQ |
| | | KSLSLSPG |
| 70 | Fc region IgG1 xELL | DKTHTCPPCPAPGGPSVFLFPPKPKDTLMISRTPEVTCVVVDV |
| | S354C, T366W knobAK447 | SHEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVMHEALHNHYTQ |
| | | KSLSLSPG |
| 101 | Fc region xELL H435R, | DKTHTCPPCPAPGGPSVFLFPPKPKDTLMISRTPEVTCVVVDV |
| | T366S, L368A, Y407V hole | SHEDPEVKFNWYVDGVEVHNAKTKPREEQYNSTYRVVSVLTVL |
| | AK447 | HQDWLNGKEYKCKVSNKALPAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNRYTQ |
| | | KSLSLSPG |
| 102 | Fc region S354C T366W | DKTHTCPPCP APELLGGPS VFLFPPKPKD TLMISRTPEV |
| | knob, AK447 | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNHYTQK SLSLSPG |
| 103 | Fc region H435R S354C | DKTHTCPPCP APELLGGPS VFLFPPKPKD TLMISRTPEV |
| | T366W knob, AK447 | TCVVVDVSHE DPEVKFNWYV DGVEVHNAKT KPREEQYNST |
| | | YRVVSVLTVL HQDWLNGKEY KCKVSNKALP APIEKTISKA |
| | | KGQPREPQVY TLPPCRDELT KNQVSLWCLV KGFYPSDIAV |
| | | EWESNGQPEN NYKTTPPVLD SDGSFFLYSK LTVDKSRWQQ |
| | | GNVFSCSVMH EALHNRYTQK SLSLSPG |
| 104 | Fc region M252Y and | DKTHTCPPCPAPELLGGPSVFLFPPKPKDTLYISRTPEVTCVV |
| | M428L (YL) S354C | VDVSHEDPEVKFNWYVDGVEVHNAKTKPREEQYNSTYRVVSVL |
| | T366W knob, AK447 | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVLHEALHNH |
| | | YTQKSLSLSPG |
| 105 | Fc region M252Y, M428L, | DKTHTCPPCPAPELLGGPSVFLFPPKPKDTLYISRTPEVTCVV |
| | H435R (YLR) S354C | VDVSHEDPEVKFNWYVDGVEVHNAKTKPREEQYNSTYRVVSVL |
| | T366W knob, AK447 | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVLHEALHNR |
| | | YTQKSLSLSPG |
| 106 | Fc region M252Y, M428V, | DKTHTCPPCPAPELLGGPSVFLFPPKPKDTLYISRTPEVTCVV |
| | H435R (YVR) S354C | VDVSHEDPEVKFNWYVDGVEVHNAKTKPREEQYNSTYRVVSVL |
| | T366W knob, AK447 | TVLHQDWLNGKEYKCKVSNKALPAPIEKTISKAKGQPREPQVY |
| | | TLPPCRDELTKNQVSLWCLVKGFYPSDIAVEWESNGQPENNYK |
| | | TTPPVLDSDGSFFLYSKLTVDKSRWQQGNVFSCSVVHEALHNR |
| | | YTQKSLSLSPG |
| 107 | Fc region T366S, L368A, | DKTHTCPPCPAPELLGGPSVFLFPPKPKDTLMISRTPEVTCVV |
| | Y407V hole, AK447 | VDVSHEDPEVKFNWYVDGVEVHNAKTKPREEQYNSTYRVVSVL |
| | | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVMHEALHNH |
| | | YTQKSLSLSPG |
| 108 | Fc region H435R, T366S, | DKTHTCPPCPAPELLGGPSVFLFPPKPKDTLMISRTPEVTCVV |
| | L368A, Y407V hole, | VDVSHEDPEVKFNWYVDGVEVHNAKTKPREEQYNSTYRVVSVL |
| | AK447 | TVLHQDWLNGKEYKCKVSNKALPAPIEKTISKAKGQPREPQVC |
| | | TLPPSRDELTKNQVSLSCAVKGFYPSDIAVEWESNGQPENNYK |
| | | TTPPVLDSDGSFFLVSKLTVDKSRWQQGNVFSCSVMHEALHNR |
| | | YTQKSLSLSPG |
| 109 | Fc region M252Y and | DKTHTCPPCPAPELLGGPSVFLFPPKPKDTLYISRTPEVTCVV |
| | M428V (YV) T366S, | VDVSHEDPEVKFNWYVDGVEVHNAKTKPREEQYNSTYRVVSVL |
| | L368A, Y407V hole, | TVLHQDWLNGKEYKCKVSNKALPAPIEKTISKAKGQPREPQVC |
| | AK447 | TLPPSRDELTKNQVSLSCAVKGFYPSDIAVEWESNGQPENNYK |
| | | TTPPVLDSDGSFFLVSKLTVDKSRWQQGNVFSCSVVHEALHNH |
| | | YTQKSLSLSPG |
| 110 | Fc region M252Y and | DKTHTCPPCPAPELLGGPSVFLFPPKPKDTLYISRTPEVTCVV |
| | M428L (YL) T366S, | VDVSHEDPEVKFNWYVDGVEVHNAKTKPREEQYNSTYRVVSVL |
| | L368A, Y407V hole, | TVLHQDWLNGKEYKCKVSNKALPAPIEKTISKAKGQPREPQVC |
| | AK447 | TLPPSRDELTKNQVSLSCAVKGFYPSDIAVEWESNGQPENNYK |
| | | TTPPVLDSDGSFFLVSKLTVDKSRWQQGNVFSCSVLHEALHNH |
| | | YTQKSLSLSPG |
| 111 | Fc region M252Y, M428L, | DKTHTCPPCPAPELLGGPSVFLFPPKPKDTLYISRTPEVTCVV |
| | H435R (YLR) T366S, | VDVSHEDPEVKFNWYVDGVEVHNAKTKPREEQYNSTYRVVSVL |
| | L368A, Y407V hole, | TVLHQDWLNGKEYKCKVSNKALPAPIEKTISKAKGQPREPQVC |
| | AK447 | TLPPSRDELTKNQVSLSCAVKGFYPSDIAVEWESNGQPENNYK |
| | | TTPPVLDSDGSFFLVSKLTVDKSRWQQGNVFSCSVLHEALHNR |
| | | YTQKSLSLSPG |
| 71 | Wild type human IL-2 | APTSSSTKKTQLQLEHLLLDLQMILNGINNYKNPKLTRMLTFK |
| | | FYMPKKATELKHLQCLEEELKPLEEVLNLAQSKNFHLRPRDLI |
| | | SNINVIVLELKGSETTEMCEYADETATIVEFLNRWITFCQSII |
| | | STLT |
| 112 | Linker 1 | GGGG |
| 113 | Linker 2 | GGSGGS |
| 114 | Linker 3 | GGSSGS |

1. A polypeptide comprising at least one VHH domain that binds CD8 and that comprises a CDR1, a CDR2, and a CDR3, respectively comprising the amino acid sequences of SEQ ID NOs: 3, 4, and 5; 3, 12, and 5; 3, 14, and 5; 3, 4, and 16; 3, 4, and 18; 3, 22, and 5; 3, 14, and 18; 3, 27, and 5; 3, 29, and 5; 3, 31, and 5; 73, 14, and 18; 74, 14, and 18; 3, 75, and 18; 3, 76, and 18; 3, 77, and 18; 3, 78, and 18; 3, 79, and 18; or 3, 80 and 18.

2. (canceled)

3. The polypeptide of claim 1, wherein at least one VHH domain comprises a CDR1 comprising the amino acid sequence of SEQ ID NO: 3; a CDR2 comprising the amino acid sequence of SEQ ID NO: 78; and a CDR3 comprising the amino acid sequence of SEQ ID NO: 18.

4. The polypeptide of claim 1, wherein at least one VHH domain, or each VHH domain, is humanized.

5. The polypeptide of claim 1, wherein at least one VHH domain comprises an amino acid sequence at least 85%, at least 90%, at least 95%, at least 99%, or 100% identical to the amino acid sequence of SEQ ID NO: 2, 6, 7, 8, 9, 10, 11, 13, 15, 17, 19, 20, 21, 23, 24, 25, 26, 28, 30, 81, 82, 83, 84, 85, 86, 87, 88, 89, 90, 91, 92, 93, 94, 95, 96, 97, 98, 99, or 100.

6. (canceled)

7. The polypeptide of claim 1, wherein at least one VHH domain comprises the amino acid sequence of SEQ ID NO: 92 or 100.

8. The polypeptide of claim 1, comprising two or three VHH domains.

9. (canceled)

10. The polypeptide of claim 1, wherein the polypeptide comprises an immune cell activating cytokine.

11. (canceled)

12. The polypeptide of claim 10, wherein the immune cell activating cytokine is IL-2, IL-15, IL-7, IL-6, IL-12, IFNα, IFNβ, or IFNγ, or an attenuated or modified version thereof.

13. The polypeptide of claim 1, wherein the polypeptide comprises an Fc region.

14. The polypeptide of claim 13, wherein the Fc region comprises an amino acid sequence selected from SEQ ID NOs: 32-70, and 101-111.

15.-17. (canceled)

18. The polypeptide of claim 1, wherein the polypeptide comprises at least one antigen-binding domain that binds an antigen other than CD8.

19. The polypeptide of claim 18, wherein the polypeptide comprises at least one antigen-binding domain that binds Lag3, CTLA4, TGFBR1, TGFBR2, Fas, TNFR2, PD1, PDL1, TIM3, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbeta1, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-L2, Phosphatidyl-serine, PIGF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.

20. (canceled)

21. The polypeptide of claim 18, wherein at least one or each antigen binding-domain that binds an antigen other than CD8 is a VHH domain or comprises a heavy chain variable region and a light chain variable region.

22.-24. (canceled)

25. A complex comprising a first polypeptide and a second polypeptide, wherein the first polypeptide is the polypeptide of claim 13, wherein the first polypeptide comprises a first Fc region, and wherein the second polypeptide comprises a second Fc region, and wherein the first and second Fc regions are the same or different.

26. The complex of claim 25, wherein the second polypeptide comprises at least one VHH domain that binds CD8, at least one immune cell activating cytokine, and/or at least one antigen binding domain that binds an antigen other than CD8.

27. (canceled)

28. The complex of claim 25, wherein the first Fc region comprises a knob mutation and the second Fc region comprises a hole mutation, or wherein the first Fc region comprises a T366W mutation and the second Fc region comprises T366S, L368A, and Y407V mutations.

29.-31. (canceled)

32. The polypeptide or complex of claim 1, wherein the CD8 is human CD8 comprising the sequence of SEQ ID NO: 1.

33. (canceled)

34. An immunoconjugate comprising the polypeptide of claim 1 and a cytotoxic agent.

35. The immunoconjugate of claim 34, wherein the cytotoxic agent is selected from a calicheamicin, an auristatin, a dolastatin, a tubulicin, a maytansinoid, a cryptophycin, a duocarmycin, an esperamicin, a pyrrolobenzodiazepine, and an enediyne antibiotic.

36. A pharmaceutical composition comprising the polypeptide of claim 1, and a pharmaceutically acceptable carrier.

37. An isolated nucleic acid that encodes the polypeptide of claim 1.

38. A vector comprising the nucleic acid of claim 37.

39. (canceled)

40. A host cell that expresses the polypeptide of claim 1.

41. A method of producing a polypeptide, comprising incubating the host cell of claim 40 under conditions suitable for expression of the polypeptide or complex.

42. (canceled)

43. A method of increasing CD8⁺ T cell proliferation comprising contacting T cells with the polypeptide of claim 1.

44. (canceled)

45. (canceled)

46. A method of treating cancer comprising administering to a subject with cancer a pharmaceutically effective amount of the polypeptide of claim 1.

47. The method of claim 46, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

48. The method of claim 46, further comprising administering an additional therapeutic agent, wherein the additional therapeutic agent is an anti-cancer agent or an anti-cancer biologic.

49. (canceled)

50. The method of claim 48, wherein the anti-cancer agent is selected from a chemotherapeutic agent, CAR-T therapy, an oncolytic virus, and an antibody.

51. (canceled)

52. The method of claim 48, wherein the anti-cancer biologic is:
(a) an agent that inhibits PD-1 and/or PD-L1;
(b) an agent that inhibits VISTA, gpNMB, B7H3, B7H4, HHLA2, CTLA4, or TIGIT; or
(c) a cytokine.

53.-57. (canceled)

58. The method of claim 46, further comprising tumor resection and/or radiation therapy.
